(19) United States
(12) Patent Application Publication
(10) Pub. No.: US 2008/0275066 A1
(43) Pub. Date: Nov. 6, 2008

(54) Condensed Thiophene Derivatives and Their Use as Cyclic Glp-1 Agonists

(75) Inventors: Sanne Moller Knudsen, Vaerlose (DK); Ingrid Pettersson, Frederiksberg (DK); Jesper Lau, Farum (DK); Carsten Behrens, Kobenhavn N (DK); Anders Klarskov Petersen, Naerum (DK); Jesper Lau, Farum (DK); Patrick William Garibay, Holte (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/630,007

(22) PCT Filed: Jun. 21, 2005
(86) PCT No.: PCT/EP05/52873

§ 371 Date: Jan. 3, 2008

(30) Foreign Application Priority Data
Jul. 2, 2004 (DK) PA 2004 01048

(51) Int. Cl. A61K 31/519; C07D 333/62; A61K 31/381; A61K 31/4365; C07D 495/04

(52) U.S. Cl. 514/260.1; 549/54; 514/443; 544/350; 546/114; 514/301

(57) ABSTRACT

The invention provides compounds of formula (I) for use as GLP-1 receptor agonists.

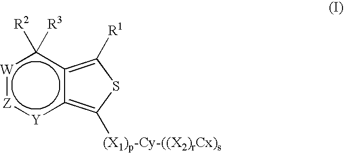

### FIELD OF THE INVENTION

The present invention relates to novel non-peptide GLP-1 agonists, pharmaceutical compositions comprising them, use of the non-peptide GLP-1 agonists for the preparation of pharmaceutical compositions and methods for the treatment and/or prevention of disorders and diseases wherein an activation of the human GLP-1 receptor is beneficial, especially metabolic disorders such as IGT (impaired glucose tolerance), Type 1 diabetes, Type 2 diabetes and obesity.

### BACKGROUND OF THE INVENTION

GLP-1 (glucagon like peptide-1) is a 30 amino acid long peptide hormone secreted by the L-cells in the intestine.

Human GLP-1 is a 37 amino acid residue peptide originating from preproglucagon which is synthesized i.a. in the L-cells in the distal ileum, in the pancreas and in the brain. GLP-1 is an important gut hormone with regulatory function in glucose metabolism and gas-trointestinal secretion and metabolism. GLP-1 stimulates insulin secretion in a glucose-dependant manner, stimulates insulin biosynthesis, promotes beta cell rescue, decreases glucagon secretion, gastric emptying and food intake.

The GLP-1 receptor is a so-called 7 transmembrane (7TM) G-protein coupled receptor. These receptors are transmembrane proteins consisting of a N-terminal extracellular part, a transmembrane core and three extracellular and three intracellular loops. The receptors are coupled to a G-protein (consisting of three subunits) and then further to an effector system. The effector system for the GLP-1 receptor is the adenylyl cyclase enzyme. Upon activation of the receptor, adenylyl cyclase catalyses the formation of the second messenger cAMP from ATP.

U.S. Pat. No. 5,670,360 to Novo Nordisk A/S discloses the cloning and use of the GLP-1 receptor. Five superfamilies of these receptors are known. Of these the glucagon-secretin (B) family consists of the receptors for GLP-1, glucagon, GIP, secretin, VIP, PACAP, calcitonin, PTH, CRF, GRF and a few more.

In prior art several GLP-1 peptides are described, however, peptides are generally not known to be orally available. The provision of orally available non-peptide GLP-1 agonists would therefore constitute a highly valuable contribution to the art.

Compounds structurally related to the present invention are described in WO98/18792, WO96/16954 and GB2336588.

### DEFINITIONS

“Halogen” designates an atom selected from the group consisting of F, Cl, Br and I.

The term “C₁₋₆-alkyl” as used herein represents a saturated, branched or straight hydrocarbon group having from 1 to 6 carbon atoms. Representative examples include, but are not limited to, methyl, ethyl, n-propyl, isopropyl, butyl, isobutyl, sec-butyl, tert-butyl, n-pentyl, isopentyl, neopentyl, tert-pentyl, n-hexyl, isohexyl and the like.

The term “C₂₋₆-alkenyl” as used herein represents a branched or straight hydrocarbon group having from 2 to 6 carbon atoms and at least one double bond. Examples of such groups include, but are not limited to, vinyl, 1-propenyl, 2-propenyl, iso-propenyl, 1,3-butadienyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 2,4-hexadienyl, 5-hexenyl and the like.

The term “C₂₋₆-alkynyl” as used herein represents a branched or straight hydrocarbon group having from 2 to 6 carbon atoms and at least one triple bond. Examples of such groups include, but are not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 2,4-hexadiynyl and the like.

The term “hydroxy-C₁₋₆alkyl” and “hydroxy-C₂₋₆-alkenyl” as used herein represents a C₁₋₆-alkyl or a C₂₋₆-alkenyl as described above, which is substituted with hydroxy.

The term “C₁₋₆-alkoxy” or “C₁₋₆-alkylsulfanyl” as used herein refers to the radical —O—C₁₋₆-alkyl and —S—C₁₋₆-alkyl respectively, wherein C₁₋₆-alkyl is as defined above. Representative examples are methoxy, ethoxy, n-propoxy, isopropoxy, butoxy, sec-butoxy, tert-butoxy, pentoxy, isopentoxy, hexoxy, isohexoxy and the corresponding thio-derivates and the like.

The term “C₁₋₆-alkanoyl” as used herein denotes a group —C(O)H or —C(O)—C₁₋₅-alkyl. Representative examples are formyl, acetyl, propionyl, butyryl, valeryl, hexanoyl and the like.

The term “C₃₋₈-cycloalkyl” as used herein represents a saturated, carbocyclic group having from 3 to 8 carbon atoms. Representative examples are cyclopropyl, cyclobutyl, cyclopentyl, cyclohexyl, cycloheptyl, cyclooctyl and the like.

The term “C₄₋₈-cycloalkenyl” as used herein represents a non-aromatic, carbocyclic group having from 4 to 8 carbon atoms containing one or two double bonds. Representative examples are 1-cyclopentenyl, 2-cyclopentenyl, 3-cyclopentenyl, 1-cyclohexenyl, 2-cyclohexenyl, 3-cyclohexenyl, 2-cycloheptenyl, 3-cycloheptenyl, 2-cyclooctenyl, 1,4-cyclooctadienyl and the like.

The term “C₃₋₈-cycloalkanoyl” as used herein represents a —C(O)—C₃₋₈-cycloalkyl, wherein C₃₋₈-cycloalkyl is as defined as above.

The term “heterocyclyl” as used herein represents a non-aromatic 3 to 10 membered ring containing one or more heteroatoms selected from nitrogen, oxygen and sulfur and optionally containing one or two double bonds. Representative examples are pyrrolidinyl, piperidyl, piperazinyl, morpholinyl, thiomorpholinyl, aziridinyl, tetrahydrofuranyl and the like.

The term “aryl” as used herein is intended to include carbocyclic aromatic ring systems such as phenyl, biphenylyl, naphthyl, anthracenyl, phenanthrenyl, fluorenyl, indenyl, pentalenyl, azulenyl and the like. Aryl is also intended to include the partially hydrogenated derivatives of the carbocyclic systems enumerated above. Non-limiting examples of such partially hydrogenated derivatives are indanyl, 1,2,3,4-tetrahydronaphthyl, 1,4-dihydronaphthyl, and the like. In an embodiment of the present invention “aryl” is selected from phenyl, naphtyl, 1,2,3,4-tetrahydronaphthyl and indanyl.

The term “arylene” as used herein is intended to include divalent carbocyclic aromatic ring systems such as phenylene, biphenylylene, naphthylene, anthracenylene, phenanthrenylene, fluorenylene, indenylene, pentalenylene, azulenylene and the like. Arylene is also intended to include the partially hydrogenated derivatives of the carbocyclic systems enumerated above. Non-limiting examples of such partially hydrogenated derivatives are 1,2,3,4-tetrahydronaphthylene, 1,4-dihydronaphthylene and the like. In an embodiment of the present invention “arylene” represents phenylene.

The term “C₁₋₆-alkyl-aryl” as used herein denotes C₁₋₆-alkyl as defined above, which has an aryl, as defined above as a substituent. Examples of this is benzyl, phenethyl, 2-phenyl-propyl, 1-phenyl-propyl etc.

The term “aryloxy” as used herein denotes a group —O-aryl, wherein aryl is as defined above.

The term “aroyl” as used herein denotes a group —C(O)-aryl, wherein aryl is as defined above.

The term “heteroaryl” as used herein is intended to include heterocyclic aromatic ring systems containing one or more heteroatoms selected from nitrogen, oxygen and sulfur such as furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuryl, benzothienyl, indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. Heteroaryl is also intended to include the partially hydrogenated derivatives of the heterocyclic systems enumerated above. Non-limiting examples of such partially hydrogenated derivatives are 2,3-dihydrobenzofuranyl, benzodioxanyl, benzoxanyl, methylenedioxybenzene, diphenyleneoxide, pyrrolinyl, pyrazolinyl, indolinyl, oxazolidinyl, oxazolinyl, oxazepinyl and the like.

In an embodiment of the present invention the term “heteroaryl” represents thienyl, thiazolyl, tetrazolyl, pyridyl, oxazolyl, 2,3-dihydrobenzofuranyl, benzodioxanyl, benzoxanyl, methylenedioxybenzene, diphenyleneoxide, pyrrolinyl, pyrazolinyl, indolinyl, oxazolidinyl, oxazolinyl, oxazepinyl

The term “optionally substituted” as used herein means that the groups in question are either unsubstituted or substituted with one or more of the substituents specified. When the groups in question are substituted with more than one substituent the substituents may be the same or different.

Some of the substituents as defined in the general formula I are divalent radicals. The above substituents are to understood as having each of the radicals attached on any suitable position in the groups mentioned above.

Certain of the above defined terms may occur more than once in the structural formulae, and upon such occurrence each term shall be defined independently of the other.

Furthermore, when using the terms “independently are” and “independently selected from” it should be understood that the groups in question may be the same or different.

### SUMMARY OF THE INVENTION

The invention provides in one aspect a compound represented by the general formula (I)

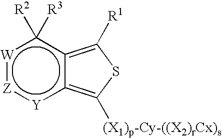

wherein the dotted circle represents optional double bonds anywhere in the ring system;
and R¹ represents —C(O)—R⁴, S(O)₂NHR⁴, C(O)N(R⁴)₂, —SR⁴ or —S(O)R⁴, or —(O)₂R⁴, wherein R⁴ is hydrogen, C₁₋₆-alkyl, C₂₋₆-alkoxy, C₂₋₆-alkenyl, C₃₋₈-cycloalkyl or C₃₋₈-cycloalkenyl; and when present twice R⁴ can be independently selected from the named substituents;
R² and R³ are independently selected from hydrogen, hydroxy, C₁₋₆-alkyl, hydroxy-C₁₋₆-alkyl C₂₋₆-alkoxy, C₂₋₆-alkylsulfanyl, —NR⁵R⁶, —N═R⁷ or the substituents attached to the same carbon atom together forms a carbonyl or thiocarbonyl group or to ═N—R⁷ or ═N—O—R⁷;
R⁵ and R⁶ are independently selected from hydrogen, C₁₋₆-alkyl, C₂₋₆-alkenyl, C₁₋₆-alkyl-aryl, aryl, C₃₋₈-cycloalkyl, C₃₋₈-cycloalkenyl, C₁₋₆-alkanoyl, aroyl, C₃₋₈-cycloalkanoyl;
R⁷ represents hydrogen, C₁₋₆-alkyl, C₂₋₆-alkenyl, aralkyl, aryl, C₃₋₈-cycloalkyl, C₃₋₈-cycloalkenyl, C₁₋₆-alkanoyl, aroyl, C₃₋₈-cycloalkanoyl, C₃₋₈-cycloalkyl, heterocyclyl, heteroaryl and arylene, wherein the rings may optionally be substituted by
   hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR⁸, —NR⁸R⁹, —SR⁸, —NR⁸S(O)₂R⁹, —S(O)₂NR⁸R⁹, —S(O)NR⁸R⁹, —S(O)R⁸, —S(O)₂R⁸, —C(O)NR⁸R⁹, —OC(O)NR⁸R⁹, —NR⁸C(O)R⁹, —CH₂C(O)NR⁸R⁹, —OCH₂C(O)NR⁸R⁹, —OC(O)R⁸, —OCH₂C(O)R , —C(O)R⁸ or —C(O)OR⁸, —OCH₂C(O)OR⁸
   C₁₋₆-alkyl, C₂₋₆-alkenyl or C₂₋₆-alkynyl,
   phenyl
which may optionally be substituted with one or more substituents selected from halogen, —CN, —CF₃, —OCF₃, —NO₂, —OR¹⁰, —NR¹⁰R¹¹ and C₁₋₆-alkyl,
wherein R₁₀ and R¹¹ independently are hydrogen, C₁₋₆-alkyl, aryl-C₁₋₆-alkyl or aryl,
or R¹⁰ and R¹¹ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds;
W, Y and Z are independently selected from NR¹², or CR¹³R¹⁴
wherein R¹² represents is hydrogen, C₁₋₆-alkyl, C₂₋₆-alkenyl, aralkyl, aryl, C₁₋₆-alkanoyl, aroyl, C₃₋₈-cycloalkanoyl, aryl;
R¹³ and R¹⁴ are independently selected from hydrogen, C₁₋₆-alkyl, C₁₋₆-alkoxy, C₁₋₆-alkylsulfanyl, or R¹³ and R¹⁴ together forms a carbonyl or thiocarbonyl; or the substituents form a double bond in the ring system; The substituents on Z and Y may optionally together form a 5- or 6-membered aromatic ring;
p, r and s are independently 0 or 1.
X₁ and X₂ each consist of A-B or B-A
wherein B is the divalent radical of the following selected from C₁₋₆-alkyl, C₂₋₆-alkoxy, C₂₋₆-alkenyl, C₂₋₆-alkynyl, hydroxy-C₁₋₆-alkyl, hydroxy-C₂₋₆-alkenyl, C₁₋₆-alkanoyl, C₂₋₆-alkenoyl;
A is selected from the group consisting of the following:

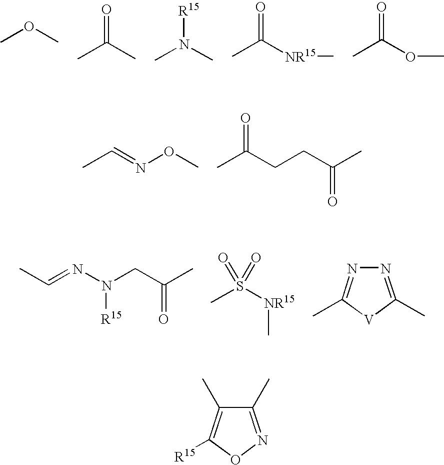

of which all may be attached to B and the ring system above in either direction;
V represents O, S, CHR¹⁵, NR¹⁵
R¹⁵ represents hydrogen, C₁₋₆-alkyl, C₂₋₆-alkenyl, C₁₋₆-alkyl-aryl, aryl, C₃₋₈-cycloalkyl, C₃₋₈-cycloalkenyl, C₁₋₆-alkanoyl, aroyl, C₃₋₈-cycloalkanoyl;
Cy and Cx are independently selected from C₃₋₈-cycloalkyl, heterocyclyl, heteroaryl and arylene, wherein the rings may optionally be substituted by
   hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR¹⁷, —NR¹⁷R¹⁸, —SR¹⁷, —NR¹⁷S(O)₂R¹⁸, —S(O)₂NR¹⁷R¹⁸, —S(O)NR¹⁷R¹⁸, —S(O)R¹⁷, —S(O)₂R¹⁷, —C(O)NR¹⁷R¹⁸, —OC(O)NR¹⁷R¹⁸, —NR¹⁷C(O)R¹⁸, —CH₂C(O)NR¹⁷R¹⁸, —OCH₂C(O)NR¹⁷R¹⁸, —OC(O)R¹⁷, —OCH₂C(O)R¹⁷, —C(O)R¹⁷ or —C(O)OR¹⁷, —OCH₂C(O)OR¹⁷
   C₁₋₆-alkyl, C₂₋₆-alkenyl or C₂₋₆-alkynyl,
   phenyl
which may optionally be further substituted with one or more substituents selected from halogen, —CN, —CF₃, —OCF₃, —NO₂, —OR¹⁷, —NR¹⁷R¹⁸ and C₁₋₆-alkyl,
wherein R¹⁷ and R¹⁸ independently are hydrogen, C₁₋₆-alkyl, aryl-C₁₋₆-alkyl or aryl,
or R¹⁷ and R¹⁸ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
wherein Cy may represent the divalent radical of any of the above;
or a pharmaceutically acceptable salt thereof;
with the proviso that when the ring system of formula I together with W, X and Y is selected to represent a 6,6-dimethyl-4,5,6,7-tetrahydrobenzo[c]thiophen-4-one and R¹ represents S—R⁴ then s and p are not simulataneously 0 and in the case of p is 1 and s is o, then X₁-Cy is not pyrrolidin-1yl-carbonyl, N-methyl-cyclohexylaminocarbonyl, homopiperidin-1-ylcarbonyl, morpholin-4-ylcarbonyl, 4-methylpiperazin-1-ylcarbonyl; and if R¹ in the same ring system is SOCH₃ and p and s are both 0 then Cy is not pyrazolyl; and if R¹ in the same ring system is SO₂CH₃ and p is 1 and s is 0 then X₁-Cy does not represent (2-methylcarboxyphenyl)-aminocarbonyl;
The invention provides compound represented by the general formula I below for use as a medicament:

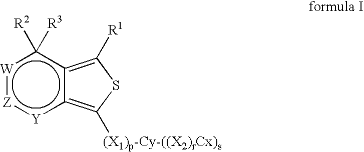

wherein the dotted circle represents optional double bonds anywhere in the ring system;
and R¹ represents —C(O)'R⁴, S(O)₂NHR⁴, C(O)N(R⁴)₂, —SR⁴ or —S(O)R⁴, or —S(O)₂R⁴, wherein R⁴ is hydrogen, C₁₋₆-alkyl, C₂₋₆-alkoxy, C₂₋₆-alkenyl, C₃₋₈-cycloalkyl or C₃₋₈-cycloalkenyl, and when present twice R⁴ can be independently selected from the named substituents;
R² and R³ are independently selected from hydrogen, hydroxy, C₁₋₆-alkyl, hydroxy-C₁₋₆-alkyl C₂₋₆-alkoxy, C₂₋₆-alkylsulfanyl, —NR⁵R⁶, —N═R⁷ or the substituents attached to the same carbon atom together forms a carbonyl or thiocarbonyl group or to ═N—R⁷ or ═N—O—R⁷;
R⁵ and R⁶ are independently selected from hydrogen, C₁₋₆-alkyl, C₂₋₆-alkenyl, C₁₋₆-alkyl-aryl, aryl, C₃₋₈-cycloalkyl, C₃₋₈-cycloalkenyl, C₁₋₆-alkanoyl, aroyl, C₃₋₈-cycloalkanoyl;
R⁷ represents hydrogen, C₁₋₆-alkyl, C₂₋₆-alkenyl, aralkyl, aryl, C₃₋₈-cycloalkyl, C₃₋₈-cycloalkenyl, C₁₋₆-alkanoyl, aroyl, C₃₋₈-cycloalkanoyl, C₃₋₈-cycloalkyl, heterocyclyl, heteroaryl and arylene, wherein the rings may optionally be substituted by
   hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR⁸, —NR⁸R⁹, —SR⁸, —NR⁸S(O)₂R⁹, —S(O)₂NR⁸R⁹, —S(O)R⁸, —S(O)₂R⁸, —C(O)NR⁸R⁹, —OC(O)NR⁸R⁹, —NR⁸C(O)R⁹, —CH₂C(O)NR⁸R⁹, —OCH₂C(O)NR⁸R⁹, —OC(O)R⁸, —OCH₂C(O)R⁸, —C(O)R⁸ or —C(O)OR⁸, —OCH₂C(O)OR⁸
   C₁₋₆-alkyl, C₂₋₆-alkenyl or C₂₋₆-alkynyl,
   phenyl
which may optionally be substituted with one or more substituents selected from halogen, —CN, —CF₃, —OCF₃, —NO₂, —OR¹⁰, —NR¹⁰R¹¹ and C₁₋₆-alkyl,
wherein R¹⁰ and R¹¹ independently are hydrogen, C_{1 6}-alkyl, aryl-C_{1 6}-alkyl or aryl,
or R¹⁰ and R¹¹ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds;
W, Y and Z are independently selected from NR¹², or CR¹³R¹⁴
wherein R¹² represents is hydrogen, C₁₋₆-alkyl, C₂₋₆-alkenyl, aralkyl, aryl, C₁₋₆-alkanoyl, aroyl, C₃₋₈-cycloalkanoyl, aryl, or the substituent forms a double bond in the ring system;
R¹³ and R¹⁴ are independently selected from hydrogen, C₁₋₆-alkyl, C₁₋₆-alkoxy, C₁₋₆-alkylsulfanyl, or R¹³ and R¹⁴ together forms a carbonyl or thiocarbonyl; or the substituents form a double bond in the ring system; The substituents on Z and Y may optionally together form a 5- or 6-membered aromatic ring;
p, r and s are independently 0 or 1.
X₁ and X₂ each consist of A-B or B-A
wherein B is the divalent radical of the following selected from C₁₋₆-alkyl, C₂₋₆-alkoxy, C₂₋₆-alkenyl, C₂₋₆-alkynyl, hydroxy-C₁₋₆-alkyl, hydroxy-C₂₋₆-alkenyl, C₁₋₆-alkanoyl, C₂₋₆-alkenoyl;
A is selected from the group consisting of the following:

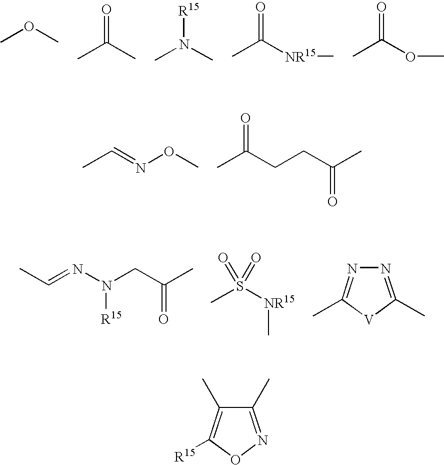

of which all may be attached to B and the ring system above in either direction;
V represents O, S, CHR¹⁵, NR¹⁵
R¹⁵ represents hydrogen, C₁₋₆-alkyl, C₂₋₆-alkenyl, C₁₋₆-alkyl-aryl, aryl, C₃₋₈-cycloalkyl, C₃₋₈-cycloalkenyl, C₁₋₆-alkanoyl, aroyl, C₃₋₈-cycloalkanoyl;
Cy and Cx are independently selected from C₃₋₈-cycloalkyl, heterocyclyl, heteroaryl and arylene, wherein the rings may optionally be substituted by
   hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR¹⁷, —NR¹⁷R¹⁸, —SR¹⁷, —NR¹⁷S(O)₂R¹⁸, —S(O)₂NR¹⁷R¹⁸, —S(O)NR¹⁷R¹⁸, —S(O)R¹⁷, —S(O)₂R¹⁷, —C(O)NR¹⁷R¹⁸, —OC(O)NR¹⁷R¹⁸, —NR¹⁷C(O)R¹⁸, —CH₂C(O)NR¹⁷R¹⁸, —OCH₂C(O)NR¹⁷R¹⁸, —OC(O)R¹⁷, —OCH₂C(O)R¹⁷, —C(O)R¹⁷ or —C(O)OR¹⁷, —OCH₂C(O)OR¹⁷
   C₁₋₆-alkyl, C₂₋₆-alkenyl or C₂₋₆-alkynyl,
   phenyl
which may optionally be further substituted with one or more substituents selected from halogen, —CN, —CF₃, —OCF₃, —NO₂, —OR¹⁷, —NR¹⁷R¹⁸ and C₁₋₆-alkyl,
wherein R¹⁷ and R¹⁸ independently are hydrogen, C₁₋₆-alkyl, aryl-C₁₋₆-alkyl or aryl,
or R¹⁷ and R¹⁸ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
wherein Cy may represent the divalent radical of any of the above;
or a pharmaceutically acceptable salt thereof;
with the proviso that when the ring system of formula I together with W, X and Y is selected to represent a 6,6-dimethyl-4,5,6,7-tetrahydrobenzo[c]thiophen-4-one and R¹ represents S—R⁴ then s and p are not simultaneously 0 and in the case of p is 1 and s is o, then X₁-Cy is not pyrrolidin-1yl-carbonyl, N-methyl-cyclohexylaminocarbonyl, homopiperidin-1-ylcarbonyl, morpholin-4-ylcarbonyl, 4-methylpiperazin-1-ylcarbonyl; and if R¹ in the same ring system is SOCH₃ and p and s are both 0 then Cy is not pyrazolyl;
The invention provides the use of a compound according to the above, for the manufacture of a medicament for the treatment and/or prevention of diseases or disorders, wherein a GLP-1 agonistic action is beneficial;
The invention provides a pharmaceutical composition comprising a compound according to any of the aspects above, together with pharmaceutically acceptable carriers and diluents.
The invention provides a method of treating or preventing a disease or disorder, wherein a GLP-1 agonistic action is beneficial, comprising administering an effective amount of a compound according to any of the aspects above.

### DETAILED DESCRIPTION OF THE INVENTION

The invention provides a compound according to the above aspect, wherein Z is NR¹²;
The invention provides a compound according to the above aspect, wherein Z is CR¹³R¹⁴;
The invention provides a compound according to any of the above aspects, wherein W is CR¹³R¹⁴;
The invention provides a compound according to any of the above aspects, wherein W is NR¹²;
The invention provides a compound according to any of the above aspects, wherein Y is CR¹³R¹⁴
The invention provides a compound according to any of the above aspects, wherein Y is NR¹²;
The invention provides a compound according to the above aspect, wherein formula (I) is

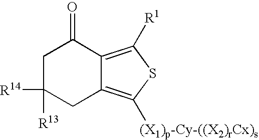

wherein R¹³, R¹⁴, R¹, X₁, X₂, Cy, Cx, p, r and s are as defined above,
with the proviso that when the ring system of formula I together with W, X and Y is selected to represent a 6,6-dimethyl-4,5,6,7-tetrahydrobenzo[c]thiophen-4-one and R¹ represents S—R⁴ then s and p are not simultaneously 0 and in the case of p is 1 and s is o, then X₁-Cy is not pyrrolidin-1yl-carbonyl, N-methyl-cyclohexylaminocarbonyl, homopiperidin-1-ylcarbonyl, morpholin-4-ylcarbonyl, 4-methylpiperazin-1-ylcarbonyl; and if R¹ in the same ring system is SOCH₃ and p and s are both 0 then Cy is not pyrazolyl; and if R¹ in the same ring system is SO₂CH₃ and p is 1 and s is 0 then X₁-Cy does not represent (2-methylcarboxyphenyl)-aminocarbonyl;
The invention provides a compound according to the above aspect, wherein formula (I) is

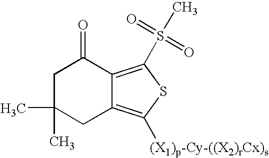

wherein X₁, X₂, Cy, Cx, p, r and s are as defined above;
with the proviso that if and p is 1 and s is 0 then X₁-Cy does not represent (2-methylcarboxyphenyl)-aminocarbonyl;
The invention provides a compound according to the above aspect, wherein formula (I) is

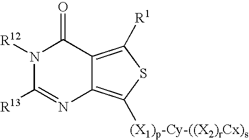

wherein R¹, R¹², X₁, X₂, Cy, Cx, p, r and s are as defined above;
The invention provides a compound according to the above aspect, wherein formula (I) is

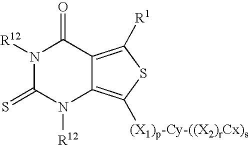

wherein R¹, R¹², X₁, X₂, Cy, Cx, p, r and s are as defined above;
The invention provides a compound according to the above aspect, wherein formula (I) is

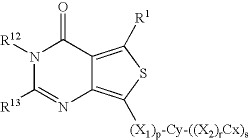

wherein R¹, R¹², R¹³, X₁, X₂, Cy, Cx, p, r and s are as defined above;
The invention provides a compound according to the above aspect, wherein R¹³ is hydrogen, C₁₋₆-alkyl, C₁₋₆-alkoxy, C₁₋₆-alkylsulfanyl
The invention provides a compound according to the above aspects, wherein R¹³ is C₁₋₆-alkylsulfanyl;
The invention provides a compound according to the above aspect, wherein formula (I) is

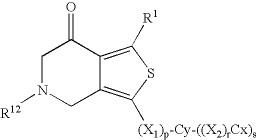

wherein R¹, R¹², X₁, X₂, Cy, Cx, p, r and s are as defined above.
The invention provides a compound according to the above aspects, wherein R¹² is C₁₋₆-alkyl, aralkyl, aryl, C₁₋₆-alkanoyl, aroyl, C₃₋₈-cycloalkanoyl, aryl
The invention provides a compound according to any of the above aspects, wherein R¹ represents —S(O)₂R⁴, wherein R⁴ is hydrogen, C₁₋₆-alkyl, C₂₋₆-alkoxy, C₂₋₆-alkoxy, C₃₋₈-cycloalkyl or C₃₋₈-cycloalkenyl;

The invention provides a compound according to the above aspect, wherein R⁴ represents C₁₋₆-alkyl;

The invention provides a compound according to any of the above aspects, wherein R⁴ represents methyl;

The invention provides a compound according to any of the above aspects, wherein A of X¹ is selected from

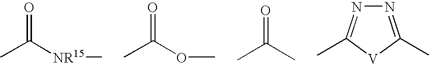

wherein R¹⁵ and V are as defined above.

The invention provides a compound according to the above aspect, wherein A of X¹ is selected from

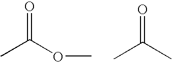

The invention provides a compound according to any of the above aspects, wherein p is 0 or 1;

The invention provides a compound according to any of the above aspects, wherein B of X¹ is C₁₋₆-alkyl;

The invention provides a compound according to the above aspect, wherein B of X¹ is selected from the group consisting of methyl, ethyl, n-propyl, isopropyl, butyl, isobutyl, sec-butyl, tert-butyl, n-pentyl, isopentyl, neopentyl, tert-pentyl, n-hexyl, isohexyl and the corresponding divalent derivatives.

The invention provides a compound according to the above aspect, wherein C₁₋₆-alkyl is selected from the group consisting of methylene, ethylene, 1,1-ethylene;

The invention provides a compound according to any of the above aspects, wherein B of X¹ is C₂₋₆-alkylene;

The invention provides a compound according to the above aspect, wherein C₂₋₆-alkenyl is selected from the group consisting of vinyl, 1-propenyl, 2-propenyl, iso-propenyl, 1,3-butadienyl, 1-butenyl, 2-butenyl, 3-butenyl, 2-methyl-1-propenyl, 1-pentenyl, 2-pentenyl 3-pentenyl, 4-pentenyl, 3-methyl-2-butenyl, 1-hexenyl, 2-hexenyl, 3-hexenyl, 2,4-hexadienyl, 5-hexenyl.

The invention provides a compound according to the above aspect, wherein C₂₋₆-alkenyl is selected from 1-propenyl, 2-propenyl or iso-propenyl;

The invention provides a compound according to any of the above aspects, wherein Cx or Cy is heteroaryl;

The invention provides a compound according to the above aspects, wherein Cx or Cy is selected from the group consisting of furyl, thienyl, pyrrolyl, 2,5-oxadiazolyl, 1,2,5-thiadiazolyl, tetrazolyl, thiazolyl, oxazolyl, isoxazolyl, isothiazolyl, pyridyl, 2,3-dihydrobenzofuranyl, benzodioxanyl, benzoxanyl, methylenedioxybenzene, diphenyleneoxide, pyrrolinyl, pyrazolinyl, indolinyl, oxazolidinyl, oxazolinyl or oxazepinyl, when Cy represents a divalent radical, then it is to include also the corresponding divalent derivatives.

The invention provides a compound according to the above aspects, wherein Cx or Cy is selected from thienyl, thiazolyl, tetrazolyl, pyridyl, oxazolyl, 2,3-dihydrobenzofuranyl, benzodioxanyl, benzoxanyl, methylenedioxybenzene, diphenyleneoxide, pyrrolinyl, pyrazolinyl, indolinyl, oxazolidinyl, oxazolinyl or oxazepinyl, and when Cy represents a divalent radical, then it is to include also the corresponding divalent derivatives;

The invention provides a compound according to the above aspects, wherein Cx or Cy is arylene or aryl;

The invention provides a compound according to the above aspects, wherein Cx or Cy is selected from the group consisting of phenylene, biphenylylene, naphthylene, anthracenylene, phenanthrenylene, fluorenylene, indenylene, pentalenylene, azulenylene,1,2,3,4-tetrahydronaphthylene, 1,4-dihydronaphthylene;

The invention provides a compound according to the above aspect, wherein Cx or Cy represents phenylene.

The invention provides a compound according to the above aspect, wherein Cx or Cy represents aryl;

The invention provides a compound according to the above aspect, wherein Cx or Cy is selected from the group consisting of phenyl, biphenylyl, naphthyl, anthracenyl, phenanthrenyl, fluorenyl, indenyl, pentalenyl, indanyl, 1,2,3,4-tetrahydronaphthyl, 1,4-dihydronaphthyl and when Cy represents a divalent radical, then it is to include also the divalent derivatives thereof;

The invention provides a compound according to the above aspect, wherein Cx or Cy is selected form the group consisting of phenyl, naphtyl, 1,2,3,4-tetrahydronaphthyl and indanyl.

The invention provides a compound according to the any of above aspects wherein Cx or Cy is C₃₋₈-cycloalkyl and when Cy represents a divalent radical, then it is to include also the divalent derivatives thereof;

The invention provides a compound according to the above aspect, wherein Cx or Cy is cyclohexyl and when Cy represents a divalent radical, then it is to include also the divalent derivatives thereof,

The invention provides a compound according to any of the above aspects, wherein Cx is phenyl, benzodioxanyl, 2-benzodioxanyl, chromanyl, indanyl, 1-indanyl, 2-indanyl, cyclohexyl, benzodioxolyl, naphtyl, oxazolyl, dibenzofuranyl, isoxazolyl, pyridyl, 1,1-dioxo-1H-benzo[b]thiophenyl, aminothiazolyl, tetrazolyl or 1,3,4-oxadiazolyl;

The invention provides a compound according to any of the above, wherein Cy is selected from the group consisting of phenyl, cyclohexyl, naphtyl, benzofuranyl, benzyl, and when Cy represents a divalent radical, then it is to include also the divalent derivatives thereof.

The invention provides a compound according to the above, wherein Cx or Cy is substituted one or more times by substituents selected independently from the group consisting of hydrogen, halogen, —CN, —CF₃, —OCF₃, —OCHF₂—NO₂, —OR⁸—C(O)OR⁸, —OCH₂C(O)OR⁸, C₁₋₆-alkyl, phenyl;

The invention provides a compound according to the above aspects, wherein X₂ is selected from the group consisting of C₁₋₆-alkyl or

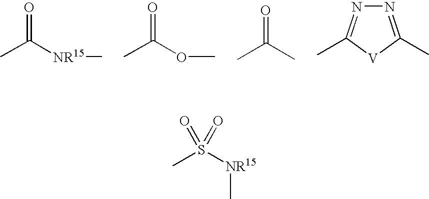

wherein R¹⁵ and V are as defined above.

The invention provides compound according to the above wherein formula I is

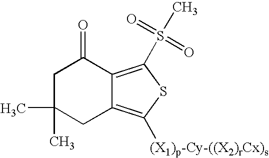

wherein X₁, X₂, Cy, Cx, p, r and s are as defined above, for use as a medicament.

The compounds of the invention may be characterised by activating the human GLP-1 receptor without necessarily competing with GLP-1 for the GLP-1 binding site in a competition binding assay.

It is believed that the compounds of the invention may stabilise another conformation of the receptor than that stabilised by GLP-1.

G-protein coupled receptors are theoretically thought to exist in different conformations: R and R*, where R is the inactive receptor conformation and R* the active.

One understanding of antagonists and inverse agonists is that they are able to bind to and stabilise the inactive conformation of the receptor whereas agonists bind to and stabilise the active conformation. It is not really known what a partial agonist does in these models.

The compounds according to the invention may introduce a new model in order to accommodate their characteristics. In this model we introduce a further receptor conformation R** which is another active receptor conformation. R* would then be the conformation that GLP-1 under normal circumstances stabilises where R** is the conformation that the compounds according to the invention stabilises. A model with two different active receptor conformations may also offer an explanation for why some of the compounds according to the invention when tested in the assays are partial and not full agonists because one conformation may be able to elicit partial agonism only and the other full agonism.

Within the context of the present invention, a GLP-1 agonist is understood to refer to any compound which fully or partially activates the human GLP-1 receptor.

Within the context of the present invention, a partial GLP-1 agonist is understood to refer to any compound which increases the activity of the human GLP-1 receptor but which compared to GLP-1 is not able to effect a full response (Eₘₐₓ<100% relative to GLP-1).

Within the context of the present invention, a GLP-1 antagonist is understood to refer to any compound which decreases the activity of the human GLP-1 receptor seen after stimulation with GLP-1.

Within the context of the present invention an inverse GLP-1 agonist is understood to refer to any compound which not only decreases the activity of the human GLP-1 receptor seen after stimulation with GLP-1 but also decreases the activity of the non-stimulated receptor (basal activity).

Within the context of the present invention a metabolic disorder is understood to refer to any disorder associated with the metabolism or resulting from a defect of the metabolism.

Within the context of the present invention GLP-1 is understood to refer to either or both of the above two native forms GLP-1 (7-36) and GLP-1 (7-37) unless otherwise specified.

Preferably, the compounds according to the invention have an EC₅₀ value as determined by the method for determining the ability to stimulate cAMP formation in a cell line expressing the cloned human GLP-1 receptor disclosed in the following of less than 25 μM, such as of less than 10 μM, more preferred of less than 2 μM and even more preferred of less than 1 μM.

In a further aspect the invention relates to a non-peptide GLP-1 agonist which activates the human GLP-1 receptor. Agonist activity may eg be determined by the assays described below.

Compounds may also be shown to be active by measuring insulin release from isolated human islets. This can be done according to the method disclosed in Eizirik D L, Korbutt G S, Hellerström C. Prolonged exposure of human pancreatic islets to high glucose concentrations in vitro impairs the beta-cell function. J. Clin. Invest. 90:1263-1268, 1992.

In a preferred embodiment the non-peptide GLP-1 agonist activates the human GLP-1 receptor without competing with GLP-1 in a competition binding assay.

In a further preferred embodiment the non-peptide GLP-1 agonist potentiates the binding of GLP-1 to the human GLP-1 receptor in a competition binding assay.

In a preferred embodiment the non-peptide GLP-1 agonist stabilises an active conformation of the human GLP-1 receptor different from the one(s) which GLP-1 stabilises.

The non-peptide GLP-1 agonists according to the invention may be either partial or full agonists.

In a further preferred embodiment the non-peptide GLP-1 agonist is a partial agonist.

Such partial agonists may be less likely of causing the receptor to desensitise because they do not fully activate the receptor and therefore also do not fully activate the desensitisation signals.

Preferably, the non-peptide partial agonists have an Eₘₐₓ of less than 90%, preferably less than 80% and more preferred in the range of 35 to 75% of that of GLP-1.

This may be determined eg by the assays described in the pharmacological methods section.

However, agonists of an Eₘₐₓ of 90% or more as well as full agonists and agonists having an Eₘₐₓ of more than 100% being efficient at lower dosages may also be usable. Thus, in another preferred embodiment the non-peptide GLP-1 agonist is a full agonist.

In still a further preferred embodiment the non-peptide GLP-1 agonist has at least a 10 fold selectivity towards the human GLP-1 receptor compared to the human glucagon receptor and/or the human GIP receptor. This may be determined eg by the assays described in the pharmacological methods section using cells expressing the human glucagon receptor and/or the human GIP receptor and comparing the formation of cAMP with the amount obtained using the cells expressing the human GLP-1 receptor.

The compounds of the present invention may have one or more asymmetric centers and it is intended that any optical isomers, as separated, pure or partially purified optical isomers or racemic mixtures thereof are included within the scope of the invention.

Furthermore, when a double bond or a fully or partially saturated ring system is present in the molecule geometric isomers may be formed. It is intended that any geometric isomers, as separated, pure or partially purified geometric isomers or mixtures thereof are included within the scope of the invention. Likewise, molecules having a bond with restricted rotation may form geometric isomers. These are also intended to be included within the scope of the present invention.

Furthermore, some of the compounds of the present invention may exist in different tautomeric forms and it is intended that any tautomeric forms which the compounds are able to form are included within the scope of the present invention.

The present invention also encompasses pharmaceutically acceptable salts of the present compounds. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

Also intended as pharmaceutically acceptable acid addition salts are the hydrates which the present compounds are able to form.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of the present invention may form solvates with standard low molecular weight solvents using methods well known to the person skilled in the art. Such solvates are also contemplated as being within the scope of the present invention.

The invention also encompasses prodrugs of the present compounds which on administration undergo chemical conversion by metabolic processes before becoming active pharmacological substances. In general, such prodrugs will be functional derivatives of the compounds of the general formula (I) which are readily convertible in vivo into the required compound of the formula (I). Conventional procedures for the selection and preparation of suitable prodrug derivatives are described, for example, in “Design of Prodrugs”, ed. H. Bundgaard, Elsevier, 1985.

The invention also encompasses active metabolites of the present compounds.

The compounds according to the present invention activate the human GLP-1 receptor and are accordingly useful for the treatment and/or prevention of disorders and diseases in which such an activation is beneficial.

Accordingly, in a further aspect the invention relates to a compound according to the invention for use as a medicament.

The invention also relates to pharmaceutical compositions comprising, as an active ingredient, at least one compound according to the invention together with one or more pharmaceutically acceptable carriers or excipients.

Furthermore, the invention relates to the use of a compound according to the invention for the preparation of a pharmaceutical composition for the treatment and/or prevention of a disorder or disease wherein an activation of the human GLP-1 receptor is beneficial.

The invention also relates to a method for the treatment and/or prevention of disorders or diseases wherein an activation of the human GLP-1 receptor is beneficial the method comprising administering to a subject in need thereof an effective amount of a compound according to the invention.

Owing to the efficiency of the present compounds to activate the human GLP-1 receptor they are useful for the treatment and/or prevention of disorders and diseases, such as metabolic disorders, wherein an activation of the said receptor is beneficial. Accordingly, they may find use in the treatment and/or prevention of hyperglycaemia, dyslipidemia, Type 1 diabetes, Type 2 diabetes, hypertriglyceridemia, syndrome X, insulin resistance, IGT, obesity, diabetes as a consequence of obesity, diabetic dyslipidemia, hyperlipidemia, cardiovascular diseases and hypertension. Furthermore, they may find use in the treatment and/or prevention of appetite regulation and energy expenditure disorders such as eating disorders eg bulimia, and other conditions where a weight reduction is required. They may also find use in the treatment and/or prevention of anxiety, movement disorder, aggression, psychosis, seizures, panic attacks, hysteria or sleep disorders. A further application is for the inhibition of intestinal motility.

In a preferred embodiment of the invention the present compounds are used for the manufacture of a medicament for the treatment and/or prevention of hyperglycemia.

In yet a preferred embodiment of the invention the present compounds are used for the manufacture of a medicament for lowering blood glucose in a mammal.

In a preferred embodiment of the invention the present compounds are used for the preparation of a pharmaceutical composition for the treatment and/or prevention of IGT.

In another preferred embodiment of the invention the present compounds are used for the preparation of a pharmaceutical composition for the treatment and/or prevention of Type 2 diabetes.

In yet another preferred embodiment of the invention the present compounds are used for the preparation of a pharmaceutical composition for the delaying or prevention of the progression from IGT to Type 2 diabetes.

In yet another preferred embodiment of the invention the present compounds are used for the preparation of a pharmaceutical composition for the delaying or prevention of the progression from non-insulin requiring Type 2 diabetes to insulin requiring Type 2 diabetes.

In a further preferred embodiment of the invention the present compounds are used for the preparation of a pharmaceutical composition for the treatment and/or prevention of Type 1 diabetes.

In a further preferred embodiment of the invention the present compounds are used for the preparation of a pharmaceutical composition for the treatment and/or prevention of obesity.

In still a further embodiment of the invention the present compounds are used for the preparation of a pharmaceutical composition for the treatment and/or prevention of an appetite regulation or energy expenditure disorder.

### Pharmaceutical Compositions

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well-known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain of from 0.05 to about 1000 mg, preferably from about 0.1 to about 500 mg, and more preferred from about 0.5 mg to about 200 mg.

For parenteral routes, such as intravenous, intrathecal, intramuscular and similar administration, typically doses are in the order of about half the dose employed for oral administration.

The compounds of this invention are generally utilized as the free substance or as a pharmaceutically acceptable salt thereof. One example is an acid addition salt of a compound having the utility of a free base. When a compound of the formula (I) contains a free base such salts are prepared in a conventional manner by treating a solution or suspension of a free base of the formula (I) with a chemical equivalent of a pharmaceutically acceptable acid, for example, inorganic and organic acids. Representative examples are mentioned above. Physiologically acceptable salts of a compound with a hydroxy group include the anion of said compound in combination with a suitable cation such as sodium or ammonium ion.

For parenteral administration, solutions of the novel compounds of the formula (I) in sterile aqueous solution, aqueous propylene glycol or sesame or peanut oil may be employed. Such aqueous solutions should be suitable buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water. Similarly, the carrier or diluent may include any sustained release material known in the art, such as glyceryl monostearate or glyceryl distearate, alone or mixed with a wax. The pharmaceutical compositions formed by combining the novel compounds of the formula (I) and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. These formulations may be in the form of powder or granules, as a solution or suspension in an aqueous or non-aqueous liquid, or as an oil-in-water or water-in-oil liquid emulsion.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet which may be prepared by conventional tabletting techniques may contain:

| | |
|---|---|
| Core: | |
| Active compound (as free compound or salt thereof) | 5.0 mg |
| Lactosum Ph. Eur. | 67.8 mg |
| Cellulose, microcryst. (Avicel) | 31.4 mg |
| Amberlite | 1.0 mg |
| Magnesii stearas Ph. Eur. | q.s. |
| Coating: | |
| HPMC approx. | 9 mg |
| Mywacett 9-40 T* approx. | 0.9 mg |

*Acylated monoglyceride used as plasticizer for film coating.

If desired, the pharmaceutical composition of the invention may comprise the compound of the formula (I) in combination with further pharmacologically active substances such as those described in the foregoing.

The present invention is further illustrated by the following representative examples which are, however, not intended to limit the scope of the invention in any way.

### EXAMPLES

Abbreviations

AcOH acetic acid

AcOEt ethyl acetate

CDI carbonyldiimidazole

DCM dichloromethane

DIAD Diisopropylazodicarboxylate

DIEA diisopropylethylamine

DMAP 4-(dimethylamino)pyridine

DMF N,N-dimethylformamide

EDAC N-Ethyl-N′-[3-(dimethylamino)propyl]carbodiimide hydrochloride

LDA Lithium diisopropylamide

mCPBA 3-chloroperbenzoic acid

MeOH methanol

PCC Pyridinium chlorochromate

rt room temperature

TBAF tetrabutyl ammonium fluoride

THF tetrahydrofuran

HPLC-MS (Method A)

The following instrumentation is used:
Hewlett Packard series 1100 G1312A Bin Pump
Hewlett Packard series 1100 Column compartment
Hewlett Packard series 1100 G13 15A DAD diode array detector
Hewlett Packard series 1100 MSD

The instrument is controlled by HP Chemstation software.

The HPLC pump is connected to two eluent reservoirs containing:

A: 0.01% TFA in water

B: 0.01% TFA in acetonitrile

The analysis is performed at 40° C. by injecting an appropriate volume of the sample (preferably 1 μl) onto the column which is eluted with a gradient of acetonitrile.

The HPLC conditions, detector settings and mass spectrometer settings used are giving in the following table.

| | |
|---|---|
| Column | Waters Xterra MS C-18 × 3 mm id |
| Gradient | 10%-100% acetonitrile lineary during 7.5 min at 1.0 ml/min |
| Detection | 210 nm (analog output from DAD) |
| MS | ionisation mode API-ES |
| | Scan 100-1000 amu step 0.1 amu |

HPLC-MS (Method B)

The following instrumentation is used:
Hewlett Packard series 1100 G1312A Bin Pump
Hewlett Packard series 1100 G13 15A DAD diode array detector
Sciex3000 triplequadropole mass spectrometer
Gilson 215 micro injector
Sedex55 evaporative light scattering detector

Pumps and detectors are controlled by MassChrom 1.1.1 software running on a Macintosh G3 computer. Gilson Unipoint Version 1.90 controls the auto-injector.

The HPLC pump is connected to two eluent reservoirs containing:

A: 0.01% TFA in water

B: 0.01% TFA in acetonitrile

The analysis is performed at room temperature by injecting an appropriate volume of the sample (preferably 10 μl) onto the column, which is eluted, with a gradient of acetonitrile. The eluate from the column passed through the UV detector to meet a flow splitter, which passed approximately 30 μl/min (1/50) through to the API Turbo ion-spray interface of API 3000 spectrometer. The remaining 1.48 ml/min (49/50) is passed through to the ELS detector.

The HPLC conditions, detector settings and mass spectrometer settings used are giving in the following table.

| | |
|---|---|
| Column | Waters X-Terra C18, 5μ, 50 mm × 3 mm id |
| Gradient | 5%-90% acetonitrile linearly during 7.5 min at 1.5 ml/min |
| Detection | 210 nm (analogue output from DAD) |
| MS | ionisation mode API Turbo ion-spray |
| | Scan 100-1000 amu step 0.1 amu |
| ELS | Gain 8 and 40° C. |

Preparation of Intermediate Compounds:

### Preparation of 1-Dihydroxymethyl-3-methanesulfonyl-6,6-dimethyl-6,7-dihydro-5H-benzo[c]thiophen-4-one

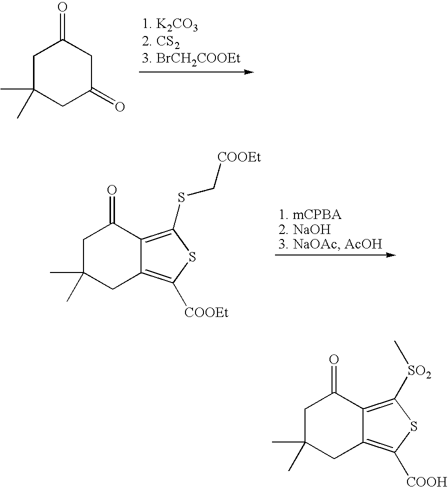

Dimedone (50 g, 357 mmol) was dissolved in DMF (500 ml) and K₂CO₃ (148 g, 1070 mmol) was added. After stirring for 10 min at rt, carbon disulfide was added (76 g, 1427 mmol). After stirring for 10 min, a solution of ethyl bromoacetate (131 g, 785 mmol) in DMF (340 ml) was added slowly using a water bath to keep the reaction at rt. After the addition, the solution was stirred for 15 min and then poured into water (4 L), and stirred for 16 h. The solution was neutralized with 1 N HCl, and the precipitate was filtered off and washed with water (3×50 ml). Drying in a vacuum oven at 40° C., yielded the orange-yellow solid, 3-ethoxycarbonylmethylsulfanyl-6,6-dimethyl-4-oxo-4,5,6,7-tetrahydrobenzo[c]thiophene-1-carboxylic acid ethyl ester (92.1 g, 70%).

3-Ethoxycarbonylmethylsulfanyl-6,6-dimethyl-4-oxo-4,5,6,7-tetrahydrobenzo[c]thiophene-1-carboxylic acid ethyl ester (92.5 g, 250 mmol) was dissolved in DCM (500 ml), and cooled to 0° C. A solution of 3-chloroperbenzoic acid (191 g, 774 mmol of 70% purity) in DCM (1.5 L) was added over 45 min while stirring at 0° C. The solution was stirred at 0° C. for 1 h and at rt for 16 h. More 3-chloroperbenzoic acid (100 g of 70% purity) in DCM (800 ml) was added and the solution was stirred for 1 d. The precipitate was filtered off and the filtrate was concentrated to approximately one half the original volume. The solution was cooled to 0° C. and a solution of Na₂SO₃ (157 g) in water (1.1 L) was added slowly. The solution was stirred vigorously for 1 h at 0° C. and for 1 h at rt. After 4 h the two phases are separated from each other and the organic phase was washed with sat. NaHCO₃ (1.5 L) and dried over MgSO₄. The solvent was removed under vacuum, and the residue was dried in a vacuum oven at 40° C. to yield a yellow oil of 3-Ethoxycarbonylmethanesulfonyl-6,6-dimethyl-4-oxo-4,5,6,7-tetrahydrobenzo[c]thiophene-1-carboxylic acid ethyl ester (83.5 g, 83% yield).

Ethoxycarbonylmethanesulfonyl-6,6-dimethyl-4-oxo-4,5,6,7-tetrahydrobenzo[c]thiophene-1-carboxylic acid ethyl ester (81 g, 202 mmol) was dissolved in THF (250 ml) and cooled to 0° C. A solution of 1 N NaOH (606 ml, 606 mmol) was added over 30 min. The reaction was stirred at a temperature of <10° C. for 45 min, removed from cooling and allowed to stir another 30 min at which time TLC (2:1 AcOEt/heptane) indicated the reaction was complete. A solution of 1 N HCl (767 ml, 767 mmol) was added. The solution was concentrated to remove the majority of the THF. The solution was extracted with ether (3×1 L). The ether extracts were pooled and dried over MgSO₄. The solvent was removed under vacuum to yield an yellow residue of 3-carboxymethylsulfonyl-6,6-dimethyl-4-oxo-4,5,6,7-tetrahydrobenzo[c]thiophene-1-carboxylic acid (68 g, 98 % yield).

3-carboxymethylsulfonyl-6,6-dimethyl-4-oxo-4,5,6,7-tetrahydrobenzo[c]thiophene-1-carboxylic acid (33.3 g, 96.2 mmol) was dissolved in acetic acid (324 ml) and sodium acetate (395 mg, 4.8 mmol) was added. After refluxing 3 h, the solvent was removed under vacuum and water (300 ml) was added. The solution was acidified with 1 N HCl (17 ml) and extracted with ether (3×400 ml). The ether phases were pooled, dried over MgSO₄ and concentrated under vacuum to yield a dark brown solid. (27 g, 94% yield). The solid was recrystallized from water (3 L) after performing a hot filtration to yield a white powder, 3-methanesulfonyl-6,6-dimethyl-4-oxo-4,5,6,7-tetrahydrobenzo[c]thiophene-1-carboxylic acid (22 g, 74% yield).

¹H-NMR (DMSO): δ 1.01 (s, 6H), 2.54 (s, 2H), 3.10 (s, 2H), 3.57 (s, 3H).

¹³C-NMR (DMSO): δ 27.67, 34.17, 42.91, 52.05, 132.44, 135.79, 149.22, 150.08, 162.18, 192.72.

MP: 207.5° C.

### Preparation of 4,4-Dimethyl-2,6-dioxo-cyclohexanecarbodithioic acid methyl ester

Method 1:

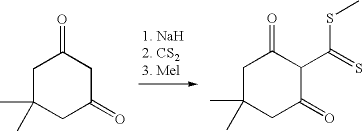

Sodium hydride (7.2 g of a 60% dispersion) was suspended in DMF (75 ml) in a flask flushed with nitrogen. The suspension was cooled to 0° C. and a solution of dimedone (20 g, 143 mmol) in DMF (75 ml) was added slowly. After stirring for 10 min at 0° C. carbon disulfide (54 g, 713 mmol) was added slowly. After stirring for 10 min at 0° C. a solution of methyl iodide (20 g, 143 mmol) in DMF (20 ml) was added slowly. The reaction mixture was poured in to a cold solution of water (1500 ml) and DCM (500 ml) was added. After acidifying with 1 N HCl, the phases were separated. The aqueous phase was extracted with DCM (2×300 ml). The organic phases were pooled, dried over MgSO₄ and concentrated. Flash chromatography (8:2 AcOEVheptane) followed by recrystallization from ethanol yielded yellow needles, 4,4-dimethyl-2,6-dioxo-cyclohexanecarbodithioic acid methyl ester (15.5 g, 47% yield).

¹H-NMR (CDCl₃, 400 MHz): δ 1.11 (s, 6H), 2.46 (br, 2H), 2.57 (s, 3H), 2.66 (br, 2H).

Method 2:

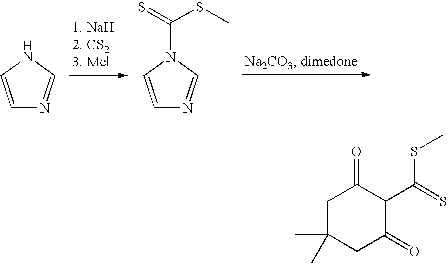

Sodium hydride (14.4 g of a 60% dispersion) was suspended in THF (125 ml). The solution was cooled to 0° C. and a solution of imidazole (34 g, 500 mmol) in THF (250 ml) was added over 15 min, and more THF (250 ml) was added. After stirring for 10 min at 0° C., a solution of carbon disulfide (46.7 g, 600 mmol) in THF (100 ml) was added over 2 min. After stirring for 30 min at 0° C., a solution of methyl iodide (90.2 g, 550 mmol) in THF (100 ml) was added, and the reaction was stirred for 40 min at 0° C. 10 ml of water was added very slowly to quench any unreacted NaH. The THF was removed under vacuum and AcOEt (500 ml) was added. The solution was washed with 5% AcOH/water and water (200 ml each), dried over Na₂SO₄, and concentrated under vacuum to yield a two-phase oil. The higher density oil was isolated using a separatory funnel to yield a yellow oil, imidazole-1-carbodithioic acid methyl ester (78.8 g, 99% yield).

¹H-NMR (CDCl₃, 400 MHz): δ 2.80 (s, 3H), 7.12 (s, 1H), 7.79 (s, 1H), 8.50 (s, 1H).

¹³C-NMR (CDCl₃, 400 MHz): δ 19.70, 117.68, 131.26, 135.56, 198.88.

Dimedone (24.9 g, 177 mmol) and imidazole-1-carbodithioic acid methyl ester (30.9 g, 177 mmol) were dissolved in 1000 ml THF, and sodium carbonate (39.4 g, 372 mmol) was added. After refluxing for 16 h, the solvent was removed under vacuum. Water (200 ml) was added, and the solution was extracted with ethyl acetate (500 ml). The aqueous phase was acidified with acetic acid (40 ml) to form a precipitated. More water (200 ml) was added, and the precipitate was filtered off and washed with water (2×100 ml) and dried under vacuum to yield a yellow powder (18.3 g). Recrystallization from ethanol (50 ml) yielded yellow crystals, 4,4-dimethyl-2,6-dioxo-cyclohexanecarbodithioic acid methyl ester (14.4 g, 35% yield).

¹H-NMR (CDCl₃, 400 MHz): δ 1.11 (s, 6H), 2.46 (s (br), 2H), 2.57 (s, 3H), 2.66 (s, (br), 2H).

### 3-Methanesulfonyl-6,6-dimethyl-4-oxo-4,5,6,7-tetrahydrobenzo[c]thiophene-1-carboxylic acid chloride

3-Methanesulfonyl-6,6-dimethyl-4-oxo-4,5,6,7-tetrahydrobenzo[c]thiophene-1-carboxylic acid (1.2 g; 3.97 mmol) was dissolved in SOCl₂ (15.0 ml, 206 mmol) and the mixture was heated to 100° C. for 4 h before the volatiles were removed in vacuo. The residue was dissolved in toluene (20 ml) and the solvent was removed in vacuo. This procedure was repeated twice. The product was dried 16 h in vacuo. Yield 1.27 g (100%) of 3-Methanesulfonyl-6,6-dimethyl-4-oxo-4,5,6,7-tetrahydrobenzo[c]thiophene-1-carboxylic acid chloride.

### Example of General Method A

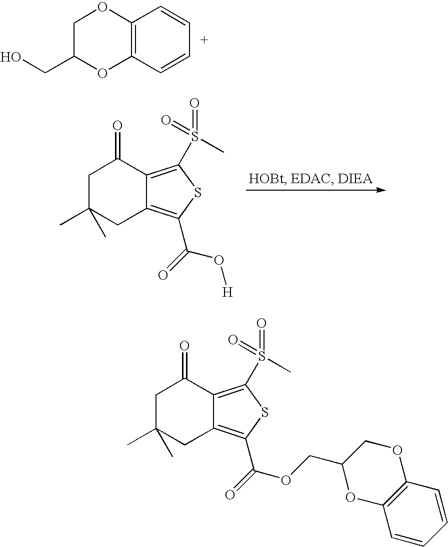

### Example 1

### 3-Methanesulfonyl-6,6-dimethyl-4-oxo-4,5,6,7-tetrahydrobenzo[c]thiophene-1-carboxylic acid 2,3-dihydro-benzo[1,4]dioxin-2-ylmethyl ester

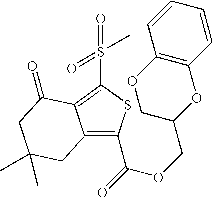

3-carboxymethanesulfonyl-6,6-dimethyl-4-oxo-4,5,6,7-tetrahydrobenzo[c]thiophene-1-carboxylic acid (440 mg, 1.46 mmol) and 1-hydroxybenzotriazole (374 mg, 2.78 mmol) were dissolved in DCM (20 ml). DIEA (358 mg, 2.77 mmol), EDAC (531 mg, 2.77 mmol) and 2-hydroxymethyl-1,4-benzodioxane (230 mg, 1.39 mmol) were added. The reaction was stirred at rt for 16 h. DCM (20 ml) was added, and the solution was washed with 5% AcOH/water, sat. NaHCO₃ and water (10 ml each). The solution was dried over MgSO₄, and concentrated under vacuum to yield a yellow powder (587 mg). Flash chromatography (Silica, 1:2 AcOEt/heptane) yielded a white solid, 3-methanesulfonyl-6,6-dimethyl-4-oxo-4,5,6,7-tetrahydrobenzo[c]thiophene-1-carboxylic acid 2,3-dihydrobenzo[1,4]dioxin-2-ylmethyl ester (435 mg, 70% yield).

¹H-NMR (400 MHz, DMSO-d₆): δ 1.00 (s, 3H) 1.01 (s, 3H) 2.57 (s, 2H) 3.11 (s, 2H) 3.59 (s, 3H) 4.12 (dd, J=11.62, 6.57 Hz, 1H) 4.43 (m, 1H) 4.60 (m, 3H) 6.87 (m, 4H).

The compounds in the following examples were prepared in a similar fashion to general method A.

### Example 2

### 3-Methanesulfonyl-6,6-dimethyl-4-oxo-4,5,6,7-tetrahydrobenzo[c]thiophene-1-carboxylic acid 3,4-dichlorobenzyl ester

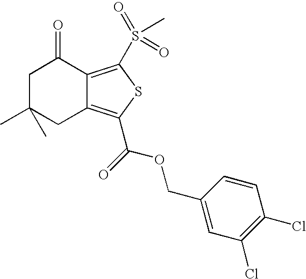

¹H-NMR (300 MHz, CDCl₃) δ 1.11 (s, 6H), 2.54 (s, 2H), 3.16 (s, 2H), 3.54 (s, 3H), 5.30 (s, 2H), 7.27 (m, 1H), 7.48 (d, J=8.29 Hz, 1H), 7.52 (d, J=2.26 Hz, 1H)

LC-MS (method A): m/z=461 (M); Rₜ=5.41 min

### Example 3

### 3-Methanesulfonyl-6,6-dimethyl-4-oxo-4,5,6,7-tetrahydrobenzo[c]thiophene-1-carboxylic acid 3-chlorobenzyl ester

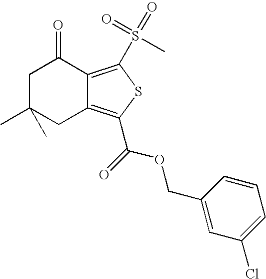

LC-MS (method A): m/z=427 (M+1); Rₜ=4.8 min

### Example 4

### 3-Methanesulfonyl-6,6-dimethyl-4-oxo-4,5,6,7-tetrahydrobenzo[c]thiophene-1-carboxylic acid 3-phenylallyl ester

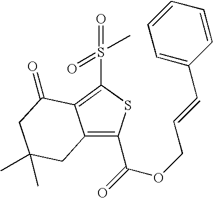

LC-MS (method A): m/z=441 (M+23); Rₜ=4.83 min

### Example 5

### 3-Methanesulfonyl-6,6-dimethyl-4-oxo-4,5,6,7-tetrahydrobenzo[c]thiophene-1-carboxylic acid 1-phenylethyl ester

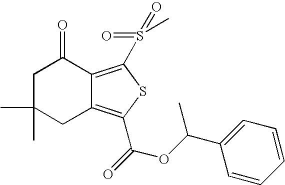

LC-MS (method A): m/z=407 (M+1); Rₜ=4.68 min

### Example 6

### 3-Methanesulfonyl-6,6-dimethyl-4-oxo-4,5,6,7-tetrahydrobenzo[c]thiophene-1-carboxylic acid 2-(thiophen-3-yl)ethyl ester

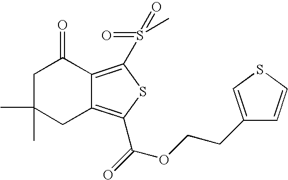

LC-MS (method A): m/z=413 (M+1); Rₜ=4.51 min

### Example 7

### 3-Methanesulfonyl-6,6-dimethyl-4-oxo-4,5,6,7-tetrahydrobenzo[c]thiophene-1-carboxylic acid 2-(thiophen-2-yl)ethyl ester

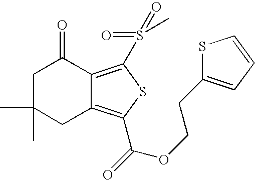

LC-MS (method A): m/z=413 (M+1); Rₜ=4.47 min

### Example 8

### 3-Methanesulfonyl-6,6-dimethyl-4-oxo-4,5,6,7-tetrahydrobenzo[c]thiophene-1-carboxylic acid chroman-4-yl ester

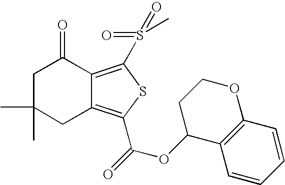

LC-MS (method A): m/z=457 (M+23); Rₜ=4.55 min

### Example 9

### 3-Methanesulfonyl-6,6-dimethyl-4-oxo-4,5,6,7-tetrahydrobenzo[c]thiophene-1-carboxylic acid indan-1-yl ester

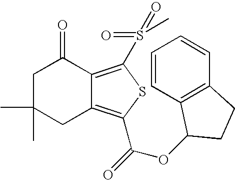

LC-MS (method A): m/z=441 (M+23); Rₜ=4.82 min

### Example 10

### 3-Methanesulfonyl-6,6-dimethyl-4-oxo-4,5,6,7-tetrahydrobenzo[c]thiophene-1-carboxylic acid 1,2,3,4-tetrahydronaphthalen-1-yl ester

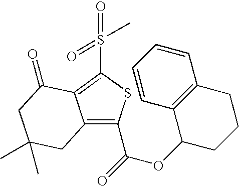

LC-MS (method A): m/z=433 (M+1); Rₜ=5.00 min

### Example 11

### 3-Methanesulfonyl-6,6-dimethyl-4-oxo-4,5,6,7-tetrahydrobenzo[c]thiophene-1-carboxylic acid thiophen-3-ylmethyl ester

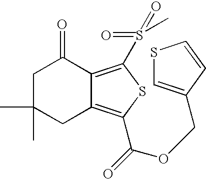

LC-MS (method A): m/z=433 (M+1); Rₜ=5.00 min

### Example 12

### 3-Methanesulfonyl-6,6-dimethyl-4-oxo-4,5,6,7-tetrahydrobenzo[c]thiophene-1-carboxylic acid indan-2-yl ester

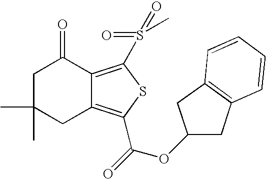

LC-MS (method A): m/z=419 (M+1); Rₜ=4.73 min

### Example 13

### 3-Methanesulfonyl-6,6-dimethyl-4-oxo-4,5,6,7-tetrahydrobenzo[c]thiophene-1-carboxylic acid 2-phenylcyclohexyl ester

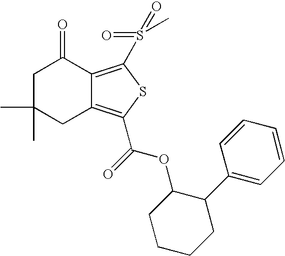

LC-MS (method A): m/z=461 (M+1); Rₜ=5.26 min

### Example 14

### 3-Methanesulfonyl-6,6-dimethyl-4-oxo-4,5,6,7-tetrahydrobenzo[c]thiophene-1-carboxylic acid 2-(4-bromophenoxy)ethyl ester

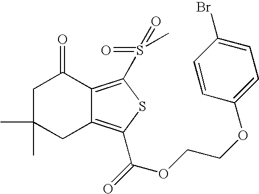

LC-MS (method A): m/z=503 (M+1); Rₜ=4.82 min

### Example 15

### 3-Methanesulfonyl-6,6-dimethyl-4-oxo-4,5,6,7-tetrahydrobenzo[c]thiophene-1-carboxylic acid 2,3-dihydro-benzo[1,4]dioxin-2-ylmethyl ester

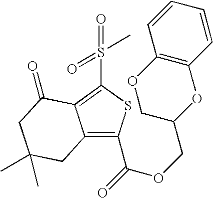

¹H-NMR (400 MHz DMSO-d₆): δ 1.00 (s, 3H) 1.01 (s, 3H) 2.57 (s, 2H) 3.11 (s, 2H) 3.59 (s, 3H) 4.12 (dd, J=11.62, 6.57 Hz, 1H) 4.43 (m, 1H) 4.60 (m, 3H) 6.87 (m, 4H).

### Example 16

### 3-Methanesulfonyl-6,6-dimethyl-4-oxo-4,5,6,7-tetrahydrobenzo[c]thiophene-1-carboxylic acid 2-trifluoromethylbenzyl ester

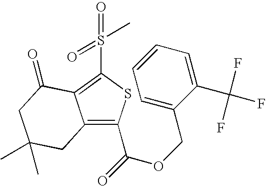

LC-MS (method A): m/z=461 (M+1); Rₜ=4.84 min

### Example 17

### 3-Methanesulfonyl-6,6-dimethyl-4-oxo-4,5,6,7-tetrahydrobenzo[c]thiophene-1-carboxylic acid 3-trifluoromethylbenzyl ester

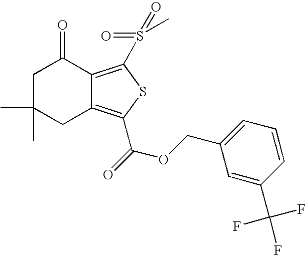

LC-MS (method A): m/z=461 (M+1); Rₜ=4.84min

### Example 18

### 3-Methanesulfonyl-6,6-dimethyl-4-oxo-4,5,6,7-tetrahydrobenzo[c]thiophene-1-carboxylic acid 2-(2-trifluoromethylphenyl)-ethyl ester

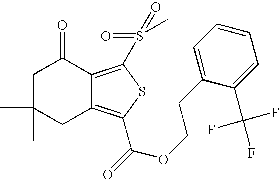

LC-MS (method A): m/z=475 (M+1); Rₜ=4.94 min

### Example 19

### 3-Methanesulfonyl-6,6-dimethyl-4-oxo-4,5,6,7-tetrahydrobenzo[c]thiophene-1-carboxylic acid benzo[1,3]dioxol-5-ylmethyl ester

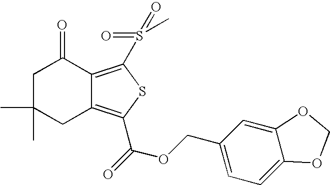

LC-MS (method A): m/z=437 (M+1); Rₜ=4.36 min

### Example 20

### 3-Methanesulfonyl-6,6-dimethyl-4-oxo-4,5,6,7-tetrahydrobenzo[c]thiophene-1-carboxylic acid (3,4-dichlorophenyl)methylamide

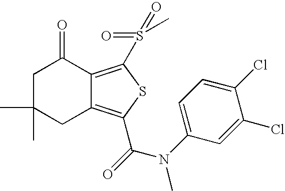

LC-MS (method A): m/z=460 (M+1); Rₜ=4.18 min

### Example 21

### 3-Methanesulfonyl-6,6-dimethyl-4-oxo-4,5,6,7-tetrahydrobenzo[c]thiophene-1-carboxylic acid 2-naphthalen-1-yl-ethyl ester

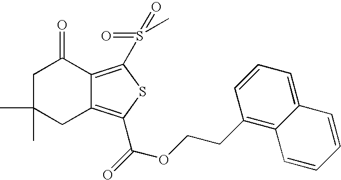

¹H-NMR (300 MHz CDCls) δ 1.55 (s, 6H), 2.51 (s, 2H), 3.07 (s, 2H), 3.54 (m, 5H), 4.67 (t, J=7.16 Hz, 2H), 7.52 (m, 4H), 7.79 (dd, J=6.41, 3.01 Hz, 1H), 7.88 (d, J=8.29 Hz, 1H), 8.11 (d, J=8.67 Hz, 1H).

LC-MS (method A): m/z=457 (M+1); Rₜ=5.02 min

### Example 22

### 3-Methanesulfonyl-6,6-dimethyl-4-oxo-4,5,6,7-tetrahydrobenzo[c]thiophene-1-carboxylic acid biphenyl-4-ylmethyl ester

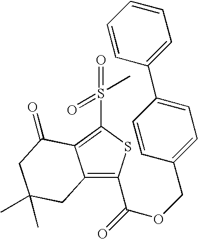

¹H-NMR (400 MHz, DMSO-d₆): δ 1.03 (s, 6H), 2.58 (s, 2H), 3.15 (s, 2H), 3.59 (s, 3H), 5.43 (s, 2H), 7.38 (t, J=7.33 Hz, 1H), 7.48 (t, J=7.58 Hz, 2H), 7.56 (d, J=8.08 Hz, 2H), 7.68 (d, J=7.07 Hz, 2H), 7.72 (d, J=8.08 Hz, 2H)

LC-MS (method A): m/z=491 (M+23); Rₜ=5.15 min

### Example 23

### 3-Methanesulfonyl-6,6-dimethyl-4-oxo-4,5,6,7-tetrahydrobenzo[c]thiophene-1-carboxylic acid 4-isopropylbenzyl ester

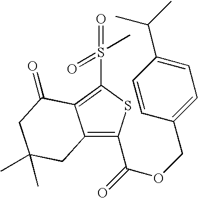

LC-MS (method A): m/z=435 (M+1); Rₜ=5.28 min

### Example 24

### 3-Methanesulfonyl-6,6-dimethyl-4-oxo-4,5,6,7-tetrahydrobenzo c]thiophene-1-carboxylic acid 3-bromobenzyl ester

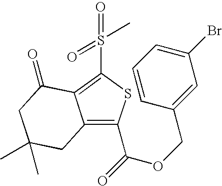

LC-MS (method A): m/z=493 (M+23); Rₜ=4.84 min

### Example 25

### 3-Methanesulfonyl-6,6-dimethyl-4-oxo-4,5,6,7-tetrahydrobenzo[c]thiophene-1-carboxylic acid 2-fluoro-4-trifluoromethylbenzyl ester

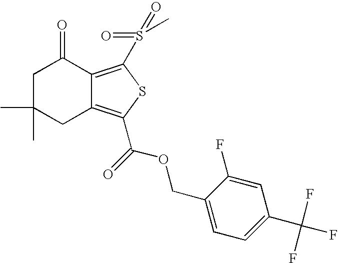

LC-MS (method A): m/z=501 (M+23); Rₜ=4.94 min

### Example 26

### 3-Methanesulfonyl-6,6-dimethyl-4-oxo-4,5,6,7-tetrahydrobenzo[c]thiophene-1-carboxylic acid 4-fluoro-2-trifluoromethylbenzyl ester

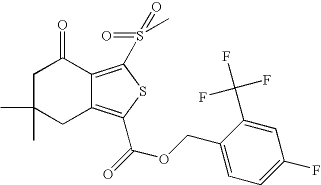

LC-MS (method A): m/z=501 (M+23); Rₜ=4.90 min

### Example 27

### 3-Methanesulfonyl-6,6-dimethyl-4-oxo-4,5,6,7-tetrahydrobenzo[c]thiophene-1-carboxylic acid 2-(5-methyl-2-phenyl-oxazol-4-yl)ethyl ester

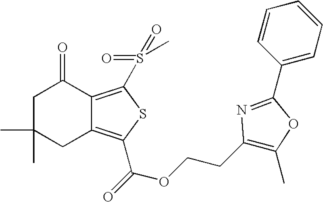

LC-MS (method A): m/z=488 (M+1); Rₜ=4.67 min

### Example 28

### 3-Methanesulfonyl-6,6-dimethyl-4-oxo-4,5,6,7-tetrahydrobenzo[c]thiophene-1-carboxylic acid 2-trifluoromethoxybenzyl ester

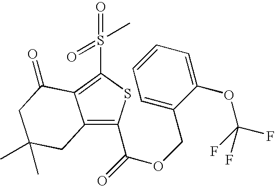

LC-MS (method A): m/z=477 (M+1); Rₜ=4.90 min

### Example 29

### 3-Methanesulfonyl-6,6-dimethyl-4-oxo-4,5,6,7-tetrahydrobenzo[c]thiophene-1-carboxylic acid 3-trifluoromethoxybenzyl ester

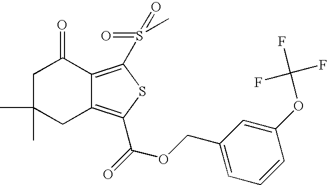

LC-MS (method A): m/z=499 (M+23); Rₜ=4.94 min

### Example 30

### 3-Methanesulfonyl-6,6-dimethyl-4-oxo-4,5,6,7-tetrahydrobenzo[c]thiophene-1-carboxylic acid 2-(3-trifluoromethylphenyl)ethyl ester

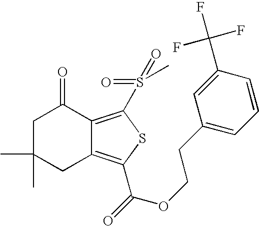

LC-MS (method A): m/z=475 (M+1); Rₜ=4.87 min

### Example 31

### 3-Methanesulfonyl-4-oxo-4,5,6,7-tetrahydrobenzo[c]thiophene-1-carboxylic acid 3,4-dichlorobenzyl ester

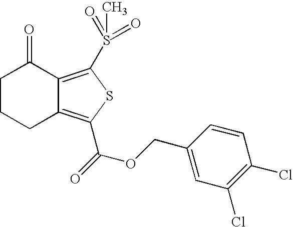

¹H-NMR (300 MHz, CDCl₃) δ 2.15 (m, 2H), 2.68 (t, 2H), 3.27 (t, 2H), 3.54 (s, 3H), 5.30 (s, 2H), 7.27 (dd, 1H), 7.47 (d, 1H), 7.51 (d, 1H)

### Example of General Method B

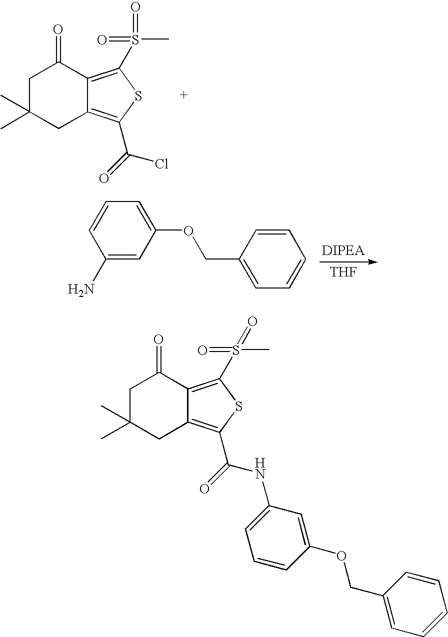

### Example 32

### 3-Methanesulfonyl-6,6-dimethyl-4-oxo-4,5,6,7-tetrahydrobenzo[c]thiophene-1-carboxylic acid (3-benzyloxyphenyl)amide

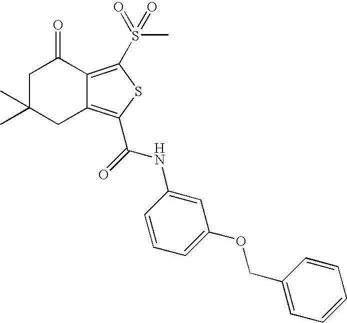

3-Benzyloxyphenylamine (31 mg, 0.156 mmol) was dissolved in THF (1 ml) and DIEA (20.1 mg, 0.156 mmol) was added. A solution of 3-methanesulfonyl-6,6-dimethyl-4-oxo-4,5,6,7-tetrahydrobenzo[c]thiophene-1-carbonyl chloride (50 mg, 0.156 mmol) in THF (1 ml) was added. The solution was mixed for 16 h, and the sample was concentrated. Water (4 ml) was added and the sample was mixed for 1 h. The precipitate was isolated by filtration to yield 3-methanesulfonyl-6,6-dimethyl-4-oxo-4,5,6,7-tetrahydrobenzo[c]thiophene-1-carboxylic acid (3-benzyloxyphenyl)amide (40 mg, 53% yield).

¹H-NMR (400 MHz) (DMSO-d₆); 10.45 (s, 1H);7.26-7.48 (m, 8H); 6.78-6.84 (m, 1H); 5.10 (s, 2H); 3.60 (s,3H); 3.06 (s, 2H); 2.58 (s, 2H); 1.04 (s, 6H).

LC-MS (method A): m/z:485 (M+1); Rₜ 4.31 min.

The compounds in the following examples were prepared in a similar fashion to general method B.

### Example 33

### 3-Methanesulfonyl-6,6-dimethyl-4-oxo-4,5,6,7-tetrahydrobenzo[c]thiophene-1-carboxylic acid (4-trifluoromethoxyphenyl)amide

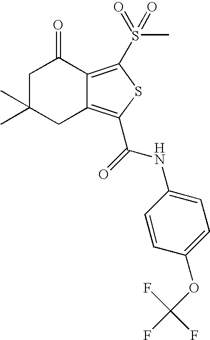

LC-MS (method A): m/z=463 (M+1); Rₜ=4,19 min.

### Example 34

### 3-Methanesulfonyl-6,6-dimethyl-4-oxo-4,5,6,7-tetrahydrobenzo[c]thiophene-1-carboxylic acid (4-benzyloxyphenyl)amide

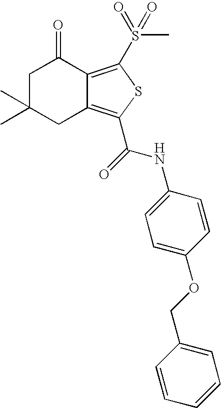

LC-MS (method A): m/z=485 (M+1); Rₜ=4.25 min.

### Example 35

### 3-Methanesulfonyl-6,6-dimethyl-4-oxo-4,5,6,7-tetrahydrobenzo[c]thiophene-1-carboxylic acid (3-phenoxyphenyl)amide

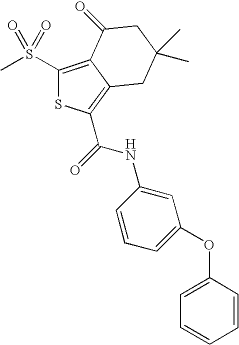

LC-MS (method A): m/z=470 (M+1); Rₜ 4.36 min.

### Example 36

### 3-Methanesulfonyl-6,6-dimethyl-4-oxo-4,5,6,7-tetrahydrobenzo[c]thiophene-1-carboxylic acid (4-cyclohexylphenyl)amide

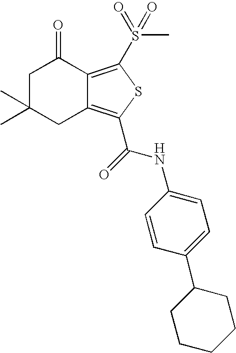

LC-MS (method A): m/z=461 (M+1); Rₜ=4.89 min.

### Example 37

### 3-Methanesulfonyl-6,6-dimethyl-4-oxo-4,5,6,7-tetrahydrobenzo[c]thiophene-1-carboxylic acid [3-(ethylphenylsulfamoyl)-4-methylphenyl]amide

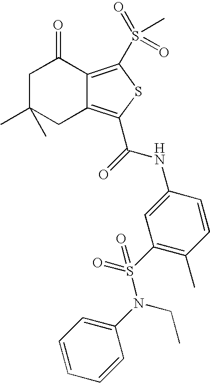

¹H-NMR (400 MHz, DMSO-d₆): 0.98-1.06 (m, 9H), 2.22 (s,3H), 2.58 (s, 2H), 3.0 (s, 2H), 3.60 (s, 3H), 3.62-3.68 (q, 2H), 7.18-7.24 (d, 2H), 7.30-7.42 (m, 4H), 7.88-7.92 (dd, 1H), 8.40-8.60 (d, 1H), 10.68 (s, 1H).

LC-MS (method A): m/z=576 (M+1); Rₜ=4.33 min.

### Example 38

### 3-Methanesulfonyl-6,6-dimethyl-4-oxo-4,5,6,7-tetrahydrobenzo[c]thiophene-1-carboxylic acid (3,4-dichlorophenyl)amide

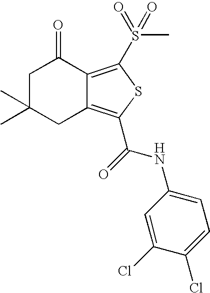

LC-MS (method A): m/z=447 (M+1); Rₜ=4.33 min.

### Example 39

### 3-Methanesufonyl-6,6-dimethyl-4-oxo-4,5,6,7-tetrahydrobenzo[c]thiophene-1-carboxylic acid (4-benzoylphenyl)amide

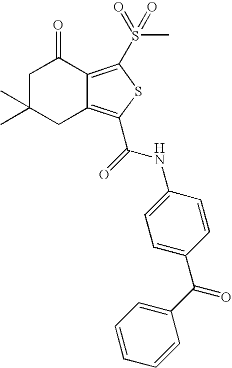

LC-MS (method A): m/z=483 (M+1); Rₜ=4.06 min.

### Example 40

### 3-Methanesulfonyl-6,6-dimethyl-4-oxo-4,5,6,7-tetrahydrobenzo[c]thiophene-1-carboxylic acid (3,5-bis-[trifluoromethyl]phenyl)amide

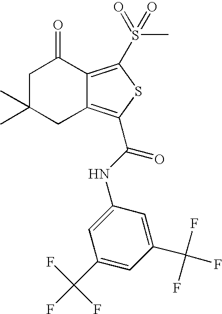

LC-MS (method A): m/z=515 (M+1); Rₜ=4.69 min.

### Example 41

### 3-Methanesulfonyl-6,6-dimethyl-4-oxo-4,5,6,7-tetrahydrobenzo[c]thiophene-1-carboxylic acid (3,5-dichlorophenyl)amide

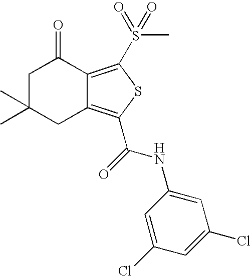

LC-MS (method A): m/z=447 (M+1); Rₜ=4.49 min.

### Example 42

### 3-Methanesulfonyl-6,6-dimethyl-4-oxo-4,5,6,7-tetrahydrobenzo[c]thiophene-1-carboxylic acid (4-tert-butylphenyl)amide

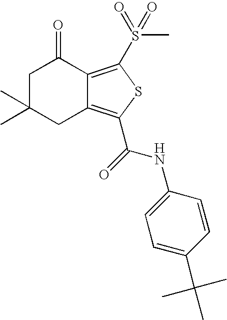

LC-MS (method A): m/z=435 (M+1); Rₜ=4.46 min.

### Example 43

### 3-Methanesulfonyl-6,6-dimethyl-4-oxo-4,5,6,7-tetrahydrobenzo[c]thiophene-1-carboxylic acid (4-chloro3-trifluoromethylphenyl)amide

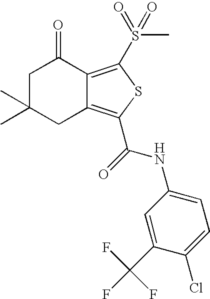

LC-MS (method A): m/z=481 (M+1); Rₜ=4.45 min.

### Example 44

### 3-Methanesulfonyl-6,6-dimethyl-4-oxo-4,5,6,7-tetrahydrobenzo[c]thiophene-1-carboxylic acid (4-secbutylphenyl)amide

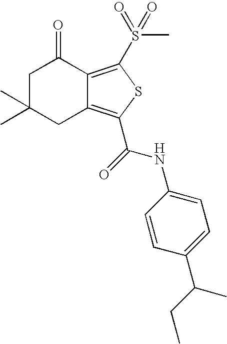

LC-MS (method A): m/z=435 (M+1); Rₜ=4.55 min.

### Example 45

### 3-Methanesulfonyl-6,6-dimethyl-4-oxo-4,5,6,7-tetrahydrobenzo[c]thiophene-1-carboxylic acid (3-benzoylphenyl)amide

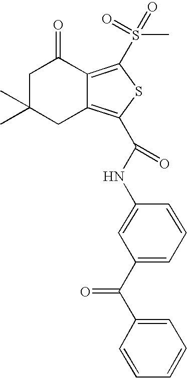

LC-MS (method A): m/z=483 (M+1); Rₜ=4.04 min.

### Example 46

### 3-Methanesulfonyl-6,6-dimethyl-4-oxo-4,5,6,7-tetrahydrobenzo[c]thiophene-1-carboxylic acid (3-tert-butylphenyl)amide

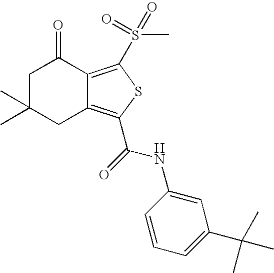

LC-MS (method A): m/z=435 (M+1); Rₜ=4.44 min.

### Example 47

### 3-Methanesulfonyl-6,6-dimethyl-4-oxo-4,5,6,7-tetrahydrobenzo[c]thiophene-1-carboxylic acid (4-phenoxyphenyl)amide

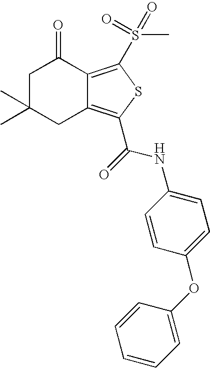

LC-MS (method A): m/z=471 (M+1); Rₜ=4.32 min.

### Example 48

### 3-Methanesulfonyl-6,6-dimethyl-4-oxo-4,5,6,7-tetrahydrobenzo[c]thiophene-1-carboxylic acid (3-methoxy-5-trifluoromethylphenyl)amide

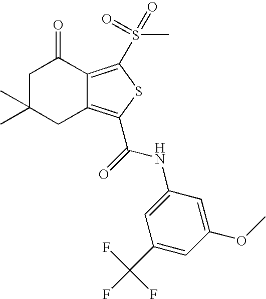

LC-MS (method A): m/z=477 (M+1); Rₜ=4.25 min.

### Example 49

### 3-Methanesulfonyl-6,6-dimethyl-4-oxo-4,5,6,7-tetrahydrobenzo[c]thiophene-1-carboxylic acid (4′-cyanobiphenyl-4-yl)amide

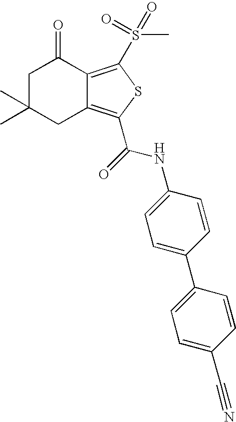

LC-MS (method A): m/z=480 (M+1); Rₜ=4.11 min.

### Example 50

### 3-Methanesulfonyl-6,6-dimethyl-4-oxo-4,5,6,7-tetrahydrobenzo[c]thiophene-1-carboxylic acid (4-butoxyphenyl)amide

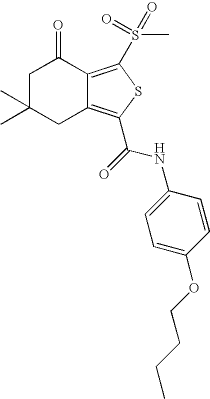

LC-MS (method A): m/z=451 (M+1); Rₜ=4.36 min.

### Example 51

### 3-Methanesulfonyl-6,6-dimethyl-4-oxo-4,5,6,7-tetrahydrobenzo[c]thiophene-1-carboxylic acid (2-methoxydibenzofuran-3-yl)amide

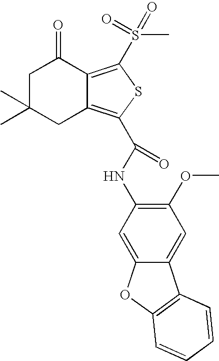

LC-MS (method A): m/z=499 (M+1); Rₜ=4.69 min.

### Example 52

### 3-Methanesulfonyl-6,6-dimethyl-4-oxo-4,5,6,7-tetrahydrobenzo[c]thiophene-1-carboxylic acid (3,5-dimethoxyphenyl)amide

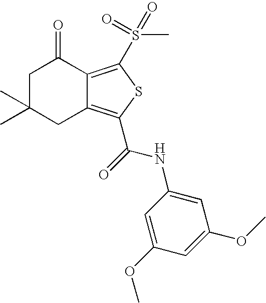

LC-MS (method A): m/z=439 (M+1); Rₜ=3.61 min.

### Example 53

### 3-Methanesulfonyl-6,6-dimethyl-4-oxo-4,5,6,7-tetrahydrobenzo[c]thiophene-1-carboxylic acid (2,2,3,3-tetrafluoro-2,3-dihydrobenzo[1,4]dioxin-6-yl)amide

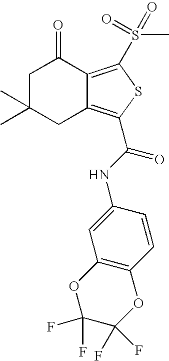

LC-MS (method A): m/z=509 (M+1); Rₜ=4.57 min.

### Example of General Method C

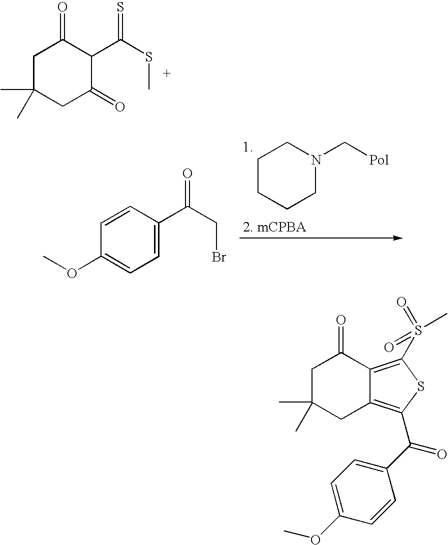

### Example 54

### 3-methanesulfonyl-1-(4-methoxybenzoyl)-6,6-dimethyl-6,7-dihydro-5H-benzo[c]thiophen-4-one

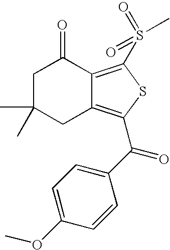

4,4-dimethyl-2,6-dioxo-cyclohexanecarbodithioic acid methyl ester (92 mg, 0.40 mmol) and 4-methoxyphenacyl bromide (92 mg, 0.40 mmol) were dissolved in 3 ml of dichloropropane, and piperidinomethyl polystyrene (336 mg, 1.20 mmol) was added. The reaction was refluxed for 16 h and the resin was filtered off, and washed with DCM (2×1 ml). The filtrate was cooled to 0° C. and 3-chloroperbenzoic acid (276 mg, 1.60 mmol of 70% purity) was added. The reaction was stirred at 0° C. for 1 h and at rt for 30 min. Water (3 ml) was added and the solution was mixed at rt for 1 h. The aqueous phase was removed, and the organic phase was washed with sat. NaHCO₃, 5% AcOH/water, and water (2 ml each). After drying over MgSO₄, the solvent was removed under vacuum to yield a residue (93 mg). After flash chromatography (silica, 1:1 AcOEt/heptane, TLC Rf=0.37) a white solid was isolated, 3-methanesulfonyl-1-(4-methoxybenzoyl)-6,6-dimethyl-6,7-dihydro-5H-benzo[c]thiophen-4-one (29 mg, 18%).

¹H-NMR (CDCl₃, 300 MHz): δ 1.10 (s, 6H), 2.57 (s, 2H), 3.02 (s, 2H), 3.57 (s, 3H), 3.92 (s, 3 H), 7.01 (d, J=9.0, 2 H), 7.01 (d, J=8.6, 2 H).

LCMS: m/z=393 (M+1) Rₜ=3.98 min.

The compounds in the following examples were prepared in a similar fashion to general method C.

### Example 55

### 1-(Biphenyl-4-carbonyl)-3-methanesulfonyl-6,6-dimethyl-6,7-dihydro-5H-benzo[c]thiophen-4-one

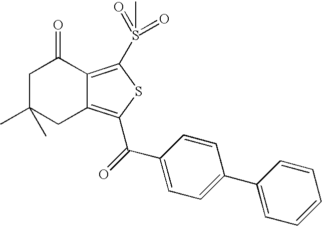

¹H-NMR (300 MHz, CDCl₃) δ 1.12 (s, 6H), 2.59 (s, 2H), 3.10 (s, 2H), 3.59 (s, 3H), 7.48 (dd, J=14.41, 7.06, 6.78 Hz, 3H), 7.66 (m, J=6.78 Hz, 2H), 7.75 (d, J=8.29 Hz, 2H), 7.95 (d, J=8.29 Hz, 2H)

LC-MS (method A): m/z=439 (M+1); Rₜ=4.84min

### Example 56

### 5-(3-Methanesulfonyl-6,6-dimethyl-4-oxo-4,5,6,7-tetrahydrobenzo[c]thiophene-1-carbonyl)isoxazole-3-carboxylic acid

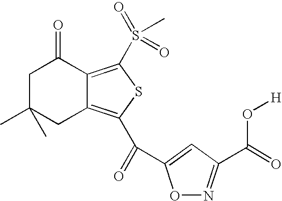

DMSO δ 1.03 (s, 6 H), 2.62 (s, 2 H), 3.12 (s, 2 H), 3.66 (s, 3 H), 7.68 (s, 1 H)

LC-MS (method A): m/z=398 (m+1) 2.84 min

### Example 57

### 1-(4-Chlorobenzoyl)-3-methanesulfonyl-6,6-dimethyl-6,7-dihydro-5H-benzo[c]thiophen-4-one

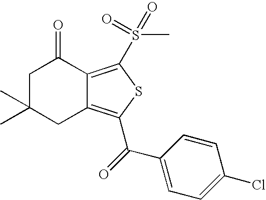

¹H-NMR (300 MHz, CDCl₃) δ 1.09 (m, 6H), 2.58 (s, 2H), 3.07 (s, 2H), 3.58 (s, 3H), 7.52 (d, J=8.67 Hz, 2H), 7.81 (d, J=8.67 Hz, 2H)

LC-MS m/z=419 (M+23); Rₜ=4.49 min.

### Example 58

### 1-(2,4-Difluorobenzoyl)-3-methanesulfonyl-6,6-dimethyl-6,7-dihydro-5H-benzo[c]thiophen-4-one

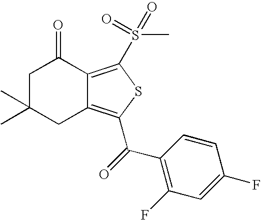

LC-MS m/z=421 (M+23); Rₜ=4.17 min.

### Example 59

### 3-Methanesulfonyl-6,6-dimethyl-1-(4-pentylbenzoyl)-6,7-dihydro-5H-benzo[c]thiophen-4-one

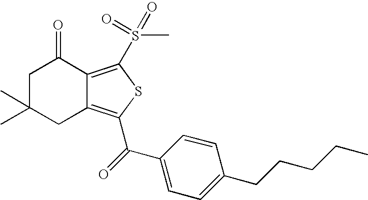

LC-MS m/z=455 (M+23); Rₜ=5.54 min.

### Example 60

### 3-Methanesulfonyl-1-(3-methoxybenzoyl)-6,6-dimethyl-6,7-dihydro-5H-benzo[c]thiophen-4-one

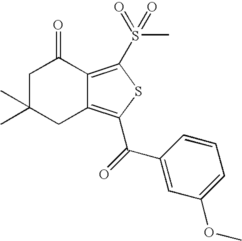

LC-MS m/z=415 (M+23); Rₜ=4.16 min.

### Example 61

### 4-(3-Methanesulfonyl-6,6-dimethyl-4-oxo-4,5,6,7-tetrahydrobenzo[c]thiophene-1-carbonyl)benzonitrile

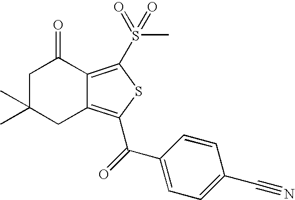

LC-MS (method B): m/z=388 (M+1); Rₜ=4.41 min.

### Example 62

### 3-Methanesulfonyl-6,6-dimethyl-1-(2-nitrobenzoyl)-6,7-dihydro-5H-benzo[c]thiophen-4-one

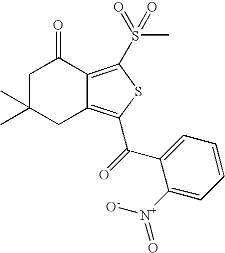

### Example 63

### 1-(4-Fluorobenzoyl)-3-methanesulfonyl-6,6-dimethyl-6,7-dihydro-5H-benzo[c]thiophen-4-one

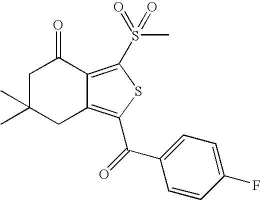

LC-MS (method B): m/z=381 (M+1); Rₜ=4.55 min.

### Example 64

### 1-(3,4-Difluorobenzoyl)-3-methanesulfonyl-6,6-dimethyl-6,7-dihydro-5H-benzo[c]thiophen-4-one

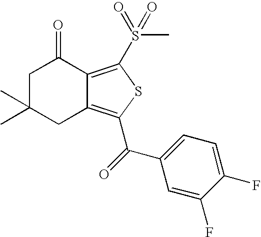

LC-MS (method B): m/z=399 (M+1); Rₜ=4.75 min.

### Example 65

### 3-Methanesulfonyl-6,6-dimethyl-1-(4-trifluoromethoxybenzoyl)-6,7-dihydro-5H-benzo[c]thiophen-4-one

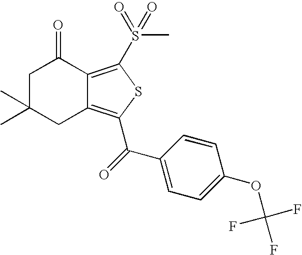

LC-MS (method B): m/z=447 (M+1); Rₜ=5.22 min.

### Example 66

### 1-(3-Fluorobenzoyl)-3-methanesulfonyl-6,6-dimethyl-6,7-dihydro-5H-benzo[c]thiophen-4-one

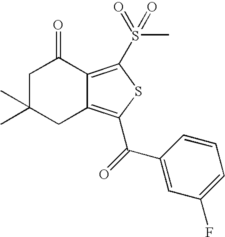

LC-MS (method B): m/z=381 (M+1); Rₜ=4.58 min.

### Example 67

### 1-(4-Difluoromethoxybenzoyl)-3-methanesulfonyl-6,6-dimethyl-6,7-dihydro-5H-benzo[c]thiophen-4-one

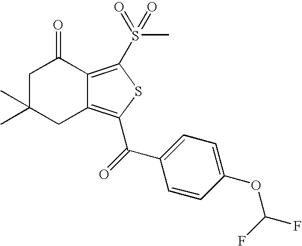

LC-MS (method B): m/z=429 (M+1 ); Rₜ=4.75 min.

### Example 68

### 3-Methanesulfonyl-1-(2-methoxybenzoyl)-6,6-dimethyl-6,7-dihydro-5H-benzo[c]thiophen-4-one

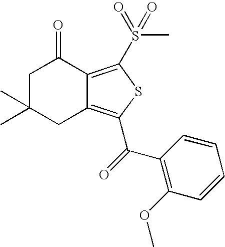

LC-MS (method B): m/z=393 (M+1); Rₜ=4.35 min.

### Example 69

### 3-Methanesulfonyl-6,6-dimethyl-1-(4-methylbenzoyl)-6,7-dihydro-5H-benzo[c]thiophen-4-one

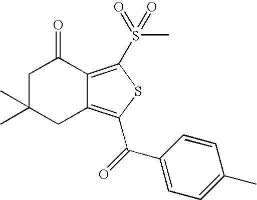

LC-MS (method B): m/z=377 (M+1); Rₜ=4.75 min.

### Example 70

### 1-[3-(4-Chlorophenyl)isoxazole-5-carbonyl]-3-methanesulfonyl-6,6-dimethyl-6,7-dihydro-5H-benzo[c]thiophen-4-one

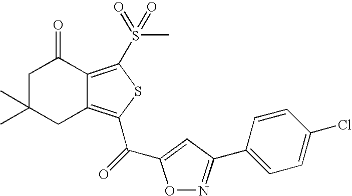

LC-MS (method B): m/z=464 (M+1); Rₜ=5.58 min.

### Example 71

### 1-(Benzo[1,3]dioxole-5-carbonyl)-3-methanesulfonyl-6,6-dimethyl-6,7-dihydro-5H-benzo[c]thiophen-4-one

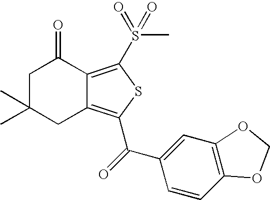

LC-MS (method B): m/z=407 (M+1); Rₜ=4.31 min.

### Example 72

### 1-(2-Chlorobenzoyl)-3-methanesulfonyl-6,6-dimethyl-6,7-dihydro-5H-benzo[c]thiophen-4-one

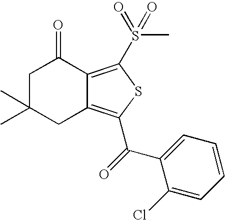

LC-MS (method B): m/z=397 (M+1); Rₜ=4.68 min.

### Example 73

### [4-(3-Methanesulfonyl-6,6-dimethyl-4-oxo-4,5,6,7-tetrahydrobenzo[c]thiophene-1-carbonyl)phenoxy]acetic acid

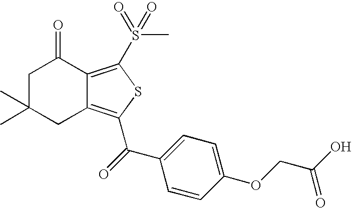

### Example 74

### 3-Methanesulfonyl-6,6-dimethyl-1-(pyridine-3-carbonyl)-6,7-dihydro-5H-benzo[c]thiophen-4-one

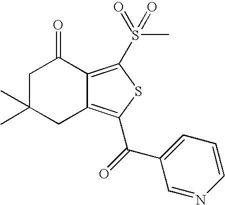

¹H-NMR (300 MHz CDCl₃) δ 1.12 (s, 6H), 2.59 (s, 2H), 3.09 (s, 2H), 3.58 (s, 3H), 7.52 (dd, J=7.91, 4.90 Hz, 1H), 8.16 (dt, J=7.91, 2.07 Hz, 1H), 8.87 (dd, J=4.90, 1.51 Hz, 1H), 9.05 (d, J=1.88 Hz, 1H)

LC-MS (method A): m/z=364 (M+1); Rₜ=3.06 min

### Example 75

### 1-(2-Fluorobenzoyl)-3-methanesulfonyl-6,6-dimethyl-6,7-dihydro-5H-benzo[c]thiophen-4-one

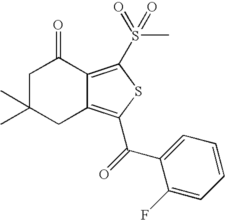

¹H-NMR (300 MHz, CDCl3) δ 1.11 (s, 6H), 2.57 (s, 2H), 3.11 (s, 2H), 3.56 (s, 3H), 7.21 (t, J=9.04 Hz, 1H), 7.31 (t, J=7.54 Hz, 1H), 7.59 (m, 2H)

LC-MS (method A): m/z=381 (M+1); Rₜ=3.75 min

### Example 76

### 1-(2,3-Dihydro-benzo[1,4]dioxine-6-carbonyl)-3-methanesulfonyl-6,6-dimethyl-6,7-dihydro-5H-benzo[c]thiophen-4-one

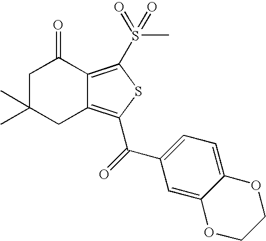

LC-MS (method B): m/z=421 (M+1); Rₜ=4.41 min.

### Example 77

### 3-Methanesulfonyl-6,6-dimethyl-1-(5-methyl-3-phenylisoxazole-4-carbonyl)-6,7-dihydro-5H-benzo[c]thiophen-4-one

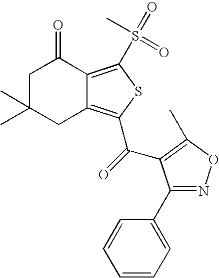

LC-MS (method B): m/z=444 (M+1); Rₜ=4.68 min.

### Example 78

### 5-(3-Methanesulfonyl-6,6-dimethyl-4-oxo-4,5,6,7-tetrahydrobenzo[c]thiophene-1-carbonyl)isoxazole-3-carboxylic acid ethyl ester

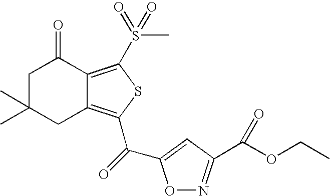

¹H-NMR (300 MHz, CDCl₃) δ 1.14 (s, 6H), 1.47 (t, J=7.16 Hz, 3H), 2.61 (s, 2H), 3.23 (s, 2H), 3.61 (s, 3H), 4.51 (q, J=7.16 Hz, 2H), 7.50 (s, 1H)

LC-MS (method A): m/z=426 (M+1); Rₜ=4.04 min.

### Example 79

### 1-(2,4-Dimethylbenzoyl)-3-methanesulfonyl-6,6-dimethyl-6,7-dihydro-5H-benzo[c]thiophen-4-one

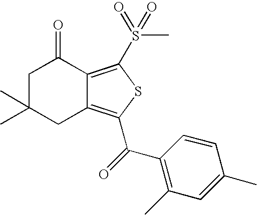

LC-MS (method B): m/z=391 (M+1); Rₜ=5.05 min.

### Example of General Method D

### Example 80

### 3-Methanesulfonyl-6,6-dimethyl-1-(4-naphthalen-2-ylthiazol-2-yl)-6,7-dihydro-5H-benzo[c]thiophen-4-one

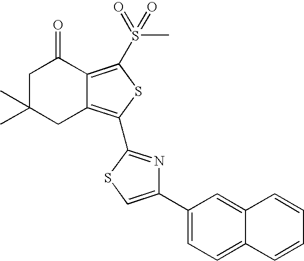

6,6-Dimethyl-3-methylthio-4-oxo-4,5,6,7-tetrahydrobenzo[c]thiophene-1-carbothioamide was obtained from commercially available 6,6-dimethyl-3-methylthio-4-oxo-4,5,6,7-tetrahydrobenzo[c]thiophene-1-carbonitrile by the procedure described previously in the literature (Chambers et al., J. Med. Chem. 2002, 45, 1176-1179).

6,6-Dimethyl-3-methylthio-4-oxo-4,5,6,7-tetrahydrobenzo[c]thiophene-1-carbothioamide (50 mg, 0.18 mmol) was suspended in ethanol (1 ml), and 2-bromo-2′-acetonaphthone (80 mg, 0.32 mmol) was added. The reaction mixture was shaken for 3 hours at 60° C. The heating was discontinued, and the solid was collected by filtration and rinsed with ethanol (0.5 ml) and ethyl acetate (0.5 ml). The crude product was dissolved in a solution of 3-chloroperoxybenzoic acid (70%,108 mg, 0.44 mmol) in dichloromethane (1 ml). The resulting reaction mixture was shaken for 1 hour at room temperature. Additional dichloromethane (2 ml) was added, and the organic phase was washed with a solution of sodium carbonate and sodium sulfite (1 M of both, 3 ml) in water. The organic layer was dried with sodium sulfate, filtered and concentrated to furnish the title compound.

LC-MS (Method A): m/z=468 (M+1); Rₜ=1.95 min.

¹H-NMR (CDCl₃): δ 8.51 (1H, s), 8.02 (1H, d), 7.94 (2H, m), 7.87 (1H, d), 7.76 (1H, s), 7.53 (2H, m), 3.58 (3H, s), 3.05 (2H, s), 2.60 (2H, s), 1.19 (6H, s).

The compounds in the following examples were prepared in a similar fashion to general method D.

### Example 81

### 3-Methanesulfonyl-6,6-dimethyl-1-[4-(3-methyl-1,1-dioxo-1H-benzo[b]thiophen-2-yl)thiazol-2-yl]-6,7-dihydro-5H-benzo[c]thiophen-4-one

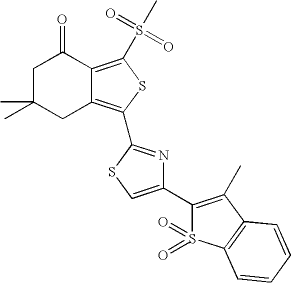

LC-MS (method B): m/z=520 (m+1); Rₜ=4.96 min

### Example 82

### 3-Methanesulfonyl-6,6-dimethyl-1-{4-[3-(3-trifluoromethylphenyl)isoxazol-5-yl]thiazol-2-yl}-6,7-dihydro-5H-benzo[c]thiophen-4-one

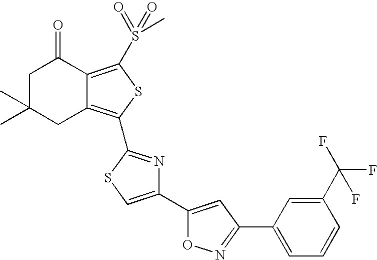

LC-MS (method B): m/z=553 (M+1); Rₜ=6.86 min

### Example 83

### 1-{4-[3-(2,6-Dichlorophenyl)-5-methylisoxazol-4-yl]thiazol-2-yl}-3-methanesulfonyl-6,6-dimethyl-6,7-dihydro-5H-benzo[c]thiophen-4-one

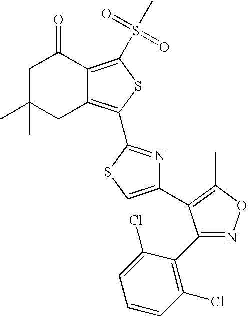

LC-MS (method B): m/z=567 (M+1); Rₜ=6.36 min

### Example 84

### 1-{4-[3-(2,4-Dichlorophenyl)isoxazol-5-yl]thiazol-2-yl}-3-methanesulfonyI-6,6-dimethyl-6,7-dihydro-5H-benzo[c]thiophen-4-one

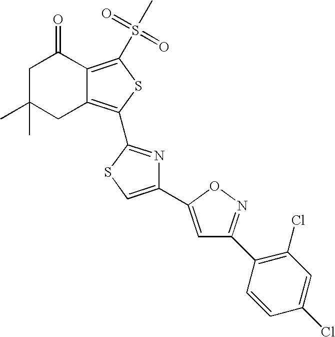

LC-MS (method B): m/z=553 (M+1); Rₜ=7.00 min

### Example 85

### 1-[4-(4-Chlorophenyl)thiazol-2-yl]-3-methanesulfonyl-6,6-dimethyl-6,7-dihydro-5H-benzo[c]thiophen-4-one

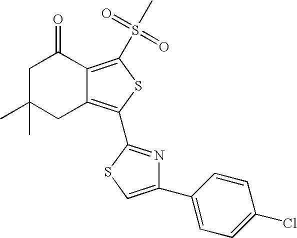

LC-MS (method B): m/z=452 (M+1); Rₜ=6.65 min

### Example 86

### 3-Methanesulfonyl-1-[4-(4-methoxyphenyl)thiazol-2-yl]-6,6-dimethyl-6,7-dihydro-5H-benzo[c]thiophen-4-one

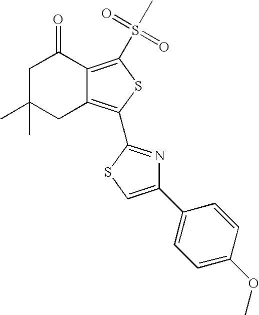

LC-MS (method B): m/z=448 (M+1); Rₜ=6.38 min

### Example 87

### 3-Methanesulfonyl-6,6-dimethyl-1-[4-(4-pentylphenyl)thiazol-2-yl]-6,7-dihydro-5H-benzo[c]thiophen-4-one

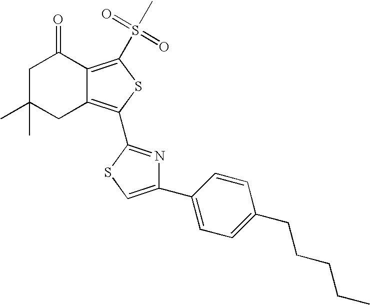

LC-MS (method B): m/z=488 (M+1); Rₜ=7.73 min

### Example 88

### 1-(4-Benzofuran-2-yl-thiazol-2-yl)-3-methanesulfonyl-6,6-dimethyl-6,7-dihydro-5H-benzo[c]thiophen-4-one

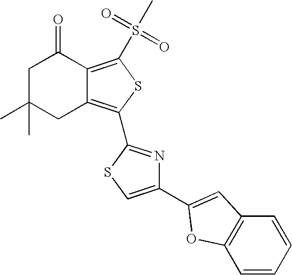

LC-MS (method B): m/z=458 (M+1); Rₜ=6.67 min

### Example 89

### 1-[4-(3,4-Dichlorophenyl)thiazol-2-yl]-3-methanesulfonyl-6,6-dimethyl-6,7-dihydro-5H-benzo[c]thiophen-4-one

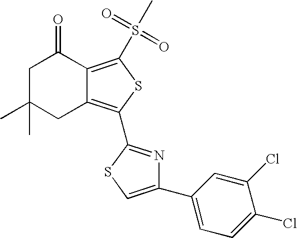

LC-MS (method B): m/z=486 (M+1); Rₜ=7.16 min

### Example 90

### 1-(4,5-Diphenylthiazol-2-yl)-3-methanesulfonyl-6,6-dimethyl-6,7-dihydro-5H-benzo[c]thiophen-4-one

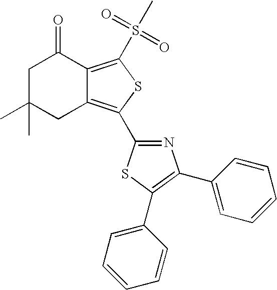

LC-MS (method B): m/z=494 (M+1); Rₜ=7.16 min

### Example 91

### 3-Methanesulfonyl-6,6-dimethyl-1-[4-(5-methyl-3-phenylisoxazol-4-yl)thiazol-2-yl]-6,7-dihydro-5H-benzo[c]thiophen-4-one

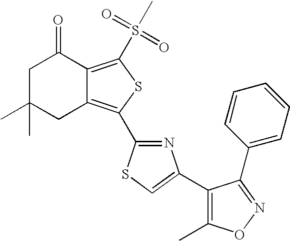

### Example 92

### 2-(3-Methanesulfonyl-6,6-dimethyl-4-oxo-4,5,6,7-tetrahydrobenzo[c]thiophen-1-yl)thiazole-4-carboxylic acid (2-fluorophenyl)amide

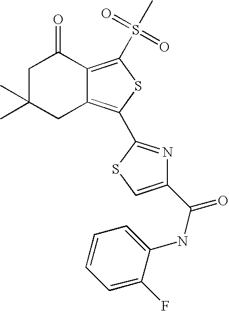

### Example of General Method E

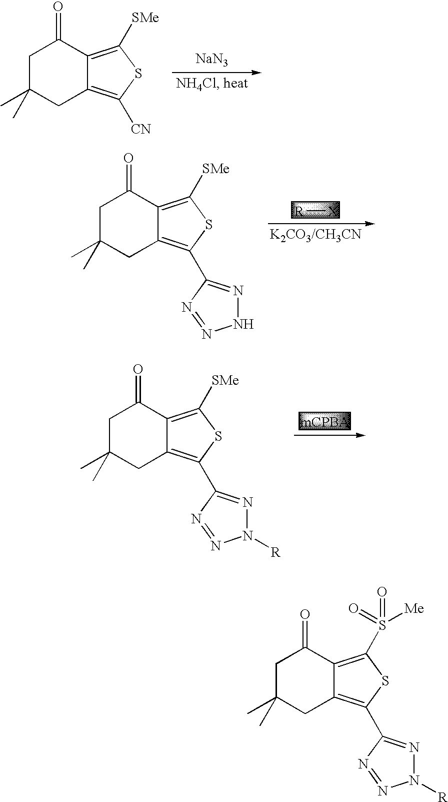

### Example 93

### 3-Methanesulfonyl-6,6-dimethyl-1-[2-(2-naphthalen-1-ylethyl)-2H-tetrazol-5-yl]-6,7-dihydro-5H-benzo[c]thiophen-4-one

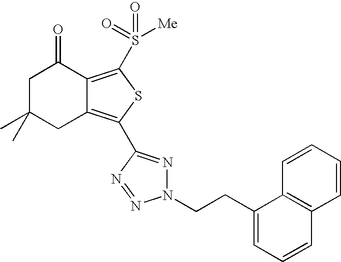

Step 1: 6,6-Dimethyl-3-methylsulfanyl-4-oxo-4,5,6,7-tetrahydrobenzo[c]thiophene-1-carbonitrile (2.00 g; 7.95 mmol) was dissolved in DMF (20 ml). Sodium azide (1.14 g; 17.5 mmol) was added followed by ammonium chloride (0.93 g; 17.5 mmol). The suspension was heated to 100° C. for 16 h. The reaction mixture was then cooled on an ice bath, diluted with water (20 ml) and acidified with hydrochloric acid (1N, 20 ml). The orange precipitate thus formed was collected by filtration, and washed twice with water, before being dried under vacuum. Yield: 2,10 g (89%).

¹H-NMR (DMSO-d₆): δ 1.01 (s, 6H); 2.42 (s, 2H); 2.65 (s, 3H); 3.03 (s, 2H).

LC-MS (method A): m/z=295 (M+1), Rₜ=2.72 min.

Step 2: 6,6-Dimethyl-3-methylsulfanyl-1-(2H-tetrazol-5-yl)-6,7-dihydro-5-benzo[c]thiophen-4-one (200 mg; 0,7 mmol) prepared as described above was dissolved in DMF (2 ml). Potassium carbonate (500 mg; 3.62 mmol) was added followed by 1-(2-bromoethyl)naphthalene (176 mg; 0,75 mmol). The suspension was heated to 100° C. for 3 h, then cooled to on an ice bath. Water (10 ml) was added, whereby a solid gum precipitated out. The water was removed by decantation, and the solid recrystallized from a minimum of ethanol, to give 3-methylthio-6,6-dimethyl-1-[2-(2-naphthalen-1-ylethyl)-2H-tetrazol-5-yl]-6,7-dihydro-5H-benzo[c]thiophen-4-one as a pure regioisomer. Yield: 93 mg (30%).

¹H-NMR (CDCl₃): δ 1.08 (s, 6H); 2.42 (s, 2H); 2.63 (s, 3H); 3.02 (s, 2H); 3.81 (t, 2H); 5.00 (t, 2H); 7.31 (d, 1H); 7.38 (d, 1H); 7.41 (d, 1H); 7.52 (t, 1H); 7.58 (t, 1H); 7.79 (d, 1H); 7.90 (d, 1H); 8.06 (d, 1H).

LC-MS (method A): m/z=449 (M+1), Rₜ=5.34 min.

Step 3: 3-methylthio-6,6-dimethyl-1-[2-(2-naphthalen-1-ylethyl)-2H-tetrazol-5-yl]-6,7-dihydro-5H-benzo[c]thiophen-4-one (50 mg; 0.11 mmol) was dissolved in DCM (1 ml) and mCPBA (58 mg; 0.335 mmol; 77% pure) was added. The mixture was stirred at ambient temperature for 1 hour, then diluted with DCM (20 ml) and washed with saturated aqueous sodium carbonate and brine. The organic phase was then dried with anhydrous sodium sulfate, and solvent removed by rotary evaporation. The residue was recrystallized from ethanol. Yield: 30 mg (60%).

¹H-NMR (DMSO-d₆): δ 1.00 (s, 6H); 2.58 (s, 2H); 2.91 (s, 2H); 3.62 (s, 3H); 3.80 (t, 2H); 5.18 (t, 2H); 7.23 (d, 1H); 7.38 (t, 1H); 7.52 (m, 2H); 7.81 (d, 1H); 7.95 (d, 1H); 8.08 (d, 1H).

LC-MS (method A): m/z=481 (M+1), Rₜ=4.48 min.

### Example 94

### 3-Methanesulfonyl-6,6-dimethyl-1-[2-(3-trifluoromethylbenzyl)-2H-tetrazol-5-yl]-6,7-dihydro-5H-benzo[c]thiophen-4-one

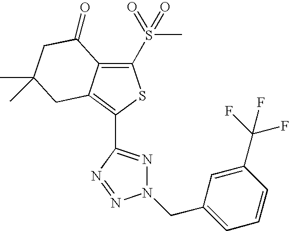

### Example of General Method F

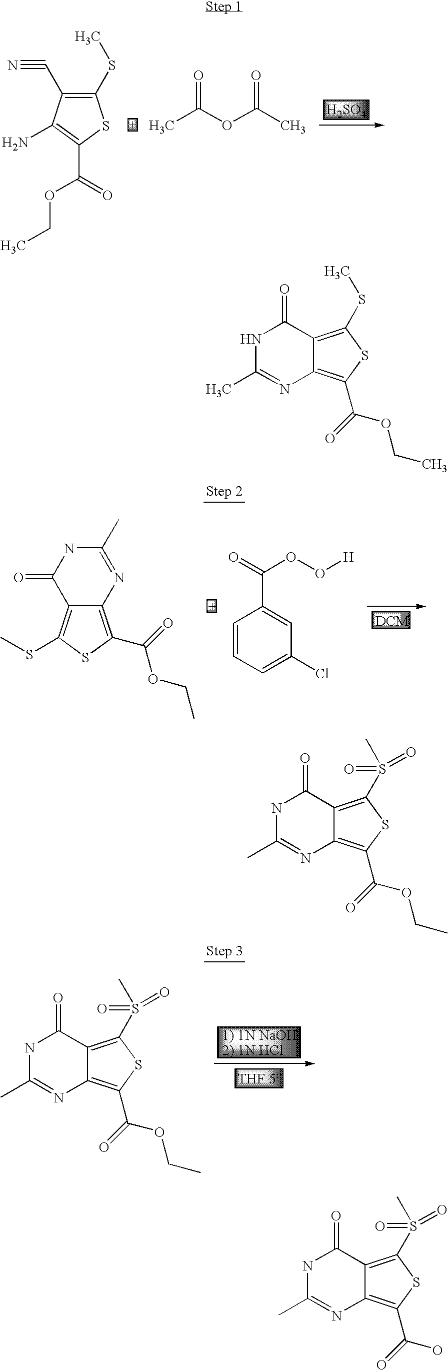

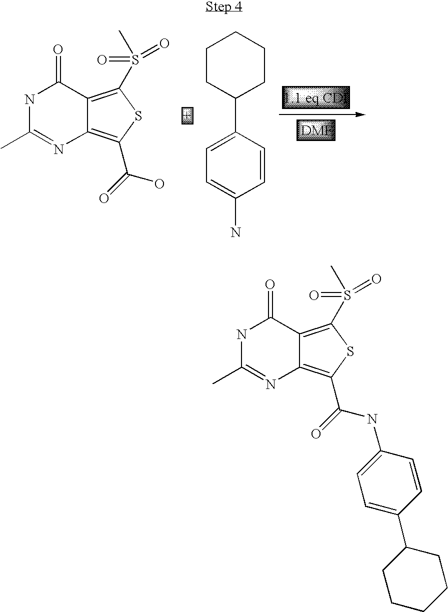

### Step 1: 2-Methyl-5-methylsulfanyl-4-oxo-3,4-thieno[3,4-d]pyrimidine-7-carboxylic acid ethyl ester

To ethyl 3-amino-cyano-5-(methylthio)thiophene-2-carboxylate (4.0 g,16, 5 mmol) was added acetic anhydride (9.0 ml, 95.2 mmol) and H₂SO₄ (0.90 ml, 96%). The temperature was raised to 100° C. and after 20 min the reaction was cooled to rt. Upon addition of NH3/MeOH (45 ml, 5M) a precipitate was formed. The precipitate was filtered off and washed with H₂O (50 ml) and dried. The precipitate was refluxed in MeOH (1 L) for 2 h. After cooling down to 5° C. the MeOH was filtered off and the precipitate was dried for 16 h in vacuo at 50° C. to give 2.7 g (57%) of 2-methyl-5-methylsulfanyl-4-oxo-3,4-thieno[3,4-d]pyrimidine-7-carboxylic acid ethyl ester.

### Step 2: 5-Methanesulfonyl-2-methyl-4-oxo-3,4-dihydro-thieno[3,4-]pyrimidine-7-carboxylic acid ethyl ester

2-Methyl-5-methylsulfanyl-4-oxo-3,4-dihydro-thieno[3,4-d]pyrimidine-7-carboxylic acid ethyl ester (2.72 g, 9.58 mmol) was suspended in DCM (50 ml). mCPBA (5.47 g, 70%, 31.68 mmol) dissolved in DCM (50 ml) was added at rt. The mixture was stirred for 30 min before Na₂SO₃ (3.98 g, 31.6 mmol) in H₂O (50 ml) was added and precipitation was observed. The suspension was vigorously stirred for 10 min whereupon the precipitate was filtered off, and washed with DCM (3×50 ml). The product was recrystallized in ethanol (200 ml) and dried in vacuo at 50° C. to give 1.7 g (56%) of 5-Methanesulfonyl-2-methyl-4-oxo-3,4-dihydro-thieno[3,4-]pyrimidine-7-carboxylic acid ethyl ester.

### Step 3: 5-Methanesulfonyl-2-methyl-4-oxo-3,4-dihydro-thieno[3,4-d]pyrimidine-7-carboxylic acid

5-Methanesulfonyl-2-methyl-4-oxo-3,4-dihydro-thieno[3,4-]pyrimidine-7-carboxylic acid ethyl ester (1.7 g, 5,37 mmol) was suspended in THF (25 ml). The reaction flask was cooled to 5° C. and NaOH (1N, 21 ml, 21 mmol) was added over the course of 5 min. The reaction mixture was stirred for 1 h at 5° C. then for 16 h at rt. The reaction mixture was cooled 5° C. and 25 ml of 1N HCl was added over 5 min (pH=2). The THF was removed in vacuo and the precipitate was filtered off and washed with H₂O (2×10 ml) and dried 16 h in vacuo to give 1.4 g (90%) of 5-methanesulfonyl-2-methyl-4-oxo-3,4-dihydrothieno[3,4-d]pyrimidine-7-carboxylic acid.

### Step 4: 5-Methanesulfonyl-2-methyl-4-oxo-3,4-dihydro-thieno[3,4-d]pyrimidine-7-carboxylic acid (4-cyclohexylphenyl)amide

5-Methanesulfonyl-2-methyl-4-oxo-3,4-dihydrothieno[3,4-d]pyrimidine-7-carboxylic acid (20 mg, 70 μmol) was dissolved in DMF (0.60 ml) and CDI (12.4 mg, 77 μmol) was added. The reaction was shaken in a glass vial for 30 min. before 4-cyclohexylanilin (13.4 mg, 77 μmol) was added. The reaction was heated with a heat gun until the starting material dissolved. The reaction mixture was shaken 12 h at rt before it was poured into H₂O (2 ml). The precipitate was filtered off and washed with H₂O (3×10 ml). The compound was dried 16 h in vacuo at 50° C. to give 15 mg (50%) of 5-methanesulfonyl-2-methyl-4-oxo-3,4-dihydro-thieno[3,4-d]pyrimidine-7-carboxylic acid (4-cyclohexylphenyl)amide.

### Example 95

### 5-Methanesulfonyl-2-methyl-4-oxo-3,4-dihydro-thieno[3,4-d]pyrimidine-7-carboxylic acid (4-cyclohexylphenyl)amide

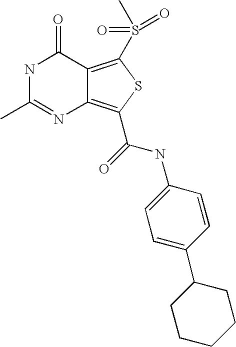

¹H-NMR (DMSO-d₆): δ 12.68 (s,1H); 11.20 (s, 1H); 7.64 (d, 2H); 7.25 (d, 2H); 3.67 (s, 3H); 1.86-1.65 (m, 5H); 1.15-1.50 (m, 5H).

¹H-NMR (CD₃OD) δ 7.6 (d, 2H); 7.23 (d, 2H); 3.61 (s, 3H); 2.53 (s, 3H); 1.9-1.7 (m, 5H); 1.53-1.2 (m, 5H).

LC-MS (method A): m/z=446 (M+1); Rₜ=4.86

### Example of General Method G

### Example 96

### 3-Methanesulfonyl-6,6-dimethyl-1-[5-(2-phenoxyphenyl)-[1,3,4]oxadiazol-2-yl]-6,7-dihydro-5H-benzo[c]thiophen-4-one

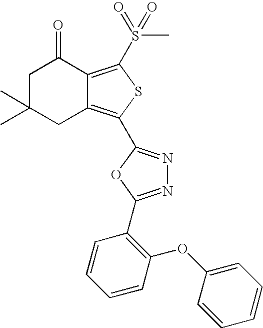

3-Methanesulfonyl-6,6-dimethyl-4-oxo-4,5,6,7-tetrahydrobenzo[c]thiophene-1-carboxylic acid chloride (50.0 mg; 156 μmol) was dissolved in THF (1 ml). 2-Phenoxybenzhydrazide (35.6 mg, 156 μmol) and DIEA (30 μl, 175 μmol) were added to the reaction. The reaction mixture was stirred for 18 h at rt before the volatiles were removed in vacuo. The residue was dissolved in toluene (3 ml) and N-benzyl-N-cyclohexylcarbodiimide-polystyrene (0.5 g) was added. The mixture was heated to 80° C. for 6 h and the solvents were removed in vacuo. Methanol (4 ml) was added and the mixture was stirred for 1 h before it was filtered. The filtrate was concentrated in vacuo to afford the crude product. The product was purified by prep. HPLC to give the pure product (77.1 mg, 49% yield) of 3-methanesulfonyl-6,6-dimethyl-1-[5-(2-phenoxyphenyl)-[1,3,4]oxadiazol-2-yl]-6,7-dihydro-5H-benzo[c]thiophen-4-one.

LC-MS (Method B): m/z=496 (M+1); Rₜ=5.38 min.

The compounds in the following examples were prepared in a similar fashion to general method G.

### Example 97

### 1-[5-(4-tert-Butylphenyl)-[1,3,4]oxadiazol-2-yl]-3-methanesulfonyl-6,6-dimethyl-6,7-dihydro-5H-benzo[c]thiophen-4-one

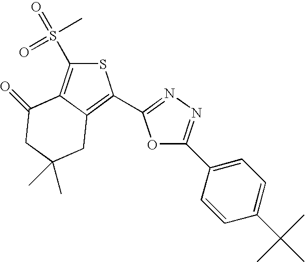

LC-MS (Method B): m/z=460 (M+1); Rₜ=5.69 min.

### Example 98

3-Methanesulfonyl-6,6-dimethyl-1-[5-(4-phenoxyphenyl)-[1,3,4]oxadiazol-2-yl]-6,7-dihydro-5H-benzo[c]thiophen-4-one

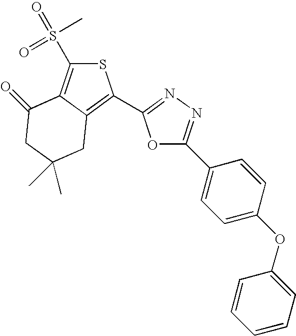

LC-MS (Method B): m/z=496 (M+1); Rₜ=5.57 min.

### Example of General Method H

4,4-Dimethyl-2,6-dioxo-cyclohexanecarbodithioic acid methyl ester (115 mg, 0.5 mmol), 4-bromomethyl-1,2-dichlorobenzene (128 mg, 0.5 mmol) and K₂CO₃ (346 mg, 2.5 mmol) were placed in a flask and acetone (5 ml) was added. The mixture was refluxed 1.5 h under nitrogen, then poured into water (35 ml). The solution was extracted with AcOEt (2×15 ml), and the organic extracts were pooled, and washed with water (plus a small amount of brine), dried over MgSO₄ and concentrated under vacuum to yield a yellow residue. The residue was dissolved in DMF (10 ml) and K₂CO₃ (346 mg, 2.5 mmol) was added. The mixture was stirred for 16 h under nitrogen at 65° C. The solution was poured into water (35 ml). The solution was extracted with AcOEt (2×15 ml), and the organic extracts were pooled, and washed with water (plus a small amount of brine), dried over MgSO₄ and concentrated under vacuum to yield a yellow residue. The product was purified by flash chromatography (silica, 2:1 heptane/AcOEt) to yield the 1-(3,4-dichlorophenyl)-6,6-dimethyl-3-methylsulfanyl-6,7-dihydro-5H-benzo[c)thiophen-4-one (32 mg).

LC-MS (Method A): m/z=371 (M+1); Rₜ=5.65 min

The 1-(3,4-dichlorophenyl)-6,6-dimethyl-3-methylsulfanyl-6,7-dihydro-5H-benzo[c)thiophen-4-one (32 mg, 0.086 mmol) was dissolved in DCM (4 ml) and cooled to 0° C. A solution of mCPBA (77 mg, 77% pure) in DCM (5 ml). The solution was stirred for 1 h at 0° and for 16 h at rt. Na₂SO₃ (109 mg, 0.862 mmol) in water (5 ml) was added and the solution was stirred vigorously for 1 h at rt. DCM (5 ml) was added and the phases were separated. The aqueous phase was extracted with DCM (5 ml). The organic extracts were pooled, dried over MgSO₄ and concentrated to yield a yellow residue (30 mg). Purification by flash chromatography (silica: 2:1 heptane/AcOEt yielded a white solid (10 mg) of 1-(3,4-dichlorophenyl)-3-methanesulfonyl-6,6-dimethyl-6,7-dihydro5H-benzo[c]thiophen-4-one.

### Example 99

### 1-(3,4-dichlorophenyl)-3-methanesulfonyl-6,6-dimethyl-6,7-dihydro5H-benzo[c]thiophen-4-one

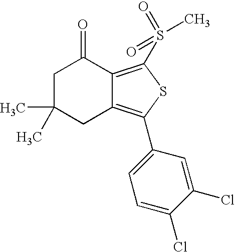

LC-MS (Method A): m/z=403 (M+1); Rₜ=4.86 min.

### Example of General Method I

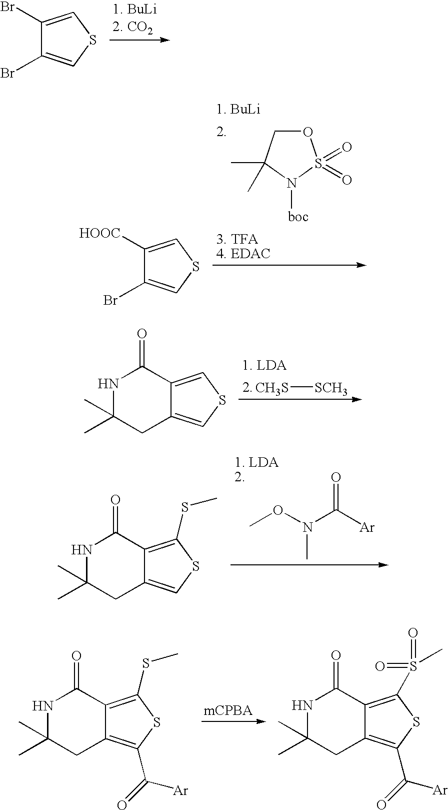

Step 1:

Preparation of 4-Bromo-thiophene-3-carboxylic acid: Ether (100 ml) was cooled to −78° C. under nitrogen. A 1.6 M solution of n-butyllithium (28.4 ml) was added. A solution of 3,4-dibromothiophene (10 g, 41.3 mmol) in 50 ml ether was added over 10 min. The solution was stirred at −78° C. for 10 min, the excess (>50 g) freshly powdered CO₂ was added. After stirring at −78° C. for 1 h, 1M NaOH (30 ml) diluted with 100 ml water was added (note: CO₂ evolution). The solution was allowed to warm until the ice melts. The phases were separated, and the ether phase was extracted with 25 ml 1 N NaOH. The aq. phases were pooled and acidified with 1N HCl (100 ml). The precipitate was filtered off and washed with water, and dried in a vacuum oven to yield white solid (5.8 g, 68% yield).

### 4-Bromo-thiophene-3-carboxylic acid

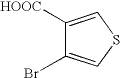

¹H-NMR (300 MHz, DMSO-d₆): δ 7.78 (s, 1H), 8.38 (s, 1H), 12.96 (br, 1H)

LC-MS (Method A): m/z=209 (M+2); Rₜ=2.12 min.

Step 2:

3-(tert-Butyloxycarbonyl)-4,4-dimethyl-[1,2,3]oxathiazolidine-2,2-dioxide can be prepared as in the literature (Posakony, J. J., Grierson, J. R. and Tewson, T. J., J. Org. Chem., 2002, 67, 5164-5169). Alternatively, the RuO₄ oxidation can be replaced by mCPBA, but the reaction is much slower, e.g. 1 month.

4-Bromo-thiophene-3-carboxylic acid is dissolved in THF, and cooled to −78° C. under nitrogen. A solution of n-butyllithium (2.2 equivalents) is then added. After stirring at −78° C. for ca. 30 min, (a solution of magnesium chloride or zinc chloride may be advantageous to add at this point) a solution of 3-(tert-butyloxycarbonyl)-4,4-dimethyl-[1,2,3]oxathiazolidine-2,2-dioxide in THF is added. The mixture is stirred for 30 min at −78° C., then allowed to warm to rt. After a standard work up, the compound can be treated with TFA or a solution of HCl in AcOEt to remove the Boc group. If the compound does not spontaneously cyclize to form the lactam, EDAC may be added to obtain the desired compound, 6,6-dimethyl-6,7-dihydro-5H-thieno[3,4-c]pyridin-4-one.

Step 3:

6,6-Dimethyl-6,7-dihydro-5H-thieno[3,4-c]pyridin-4-one is dissolved in THF, and cooled to −78° C. under nitrogen. 2.2 equivalents of a solution of LDA are then added. After stirring at −78° C. for ca. 30 min, (a solution of magnesium chloride or zinc chloride may be advantageous to add at this point) a solution of dimethyl disulfide in THF is then added. The mixture is stirred for 30 min at −78° C., then allow to warm to rt. After a standard work up and purification the desired product can be isolated, 6,6-dimethyl-3-methylsulfanyl-6,7-dihydro-5H-thieno[3,4-c]pyridin-4-one.

Step 4:

The N-methoxy-N-methyl arylamides can be prepared in several standard ways, one example is shown below for 2-fluoro-N-methoxy-N-methyl-benzamide.

N,O-Dimethylhydroxylamine hydrochloride (23.4 g, 240 mmol) was suspended in THF (100 ml) and cooled to 0° C. under nitrogen. Pyridine (32 ml, 400 mmol) was added slowly. A solution of 2-fluorobenzoyl chloride (9.5 ml, 80 mmol) in THF (50 ml) was added over 15 min. The reaction was removed from the ice bath and stirred at rt for 2 h. Water (100 ml) and AcOEt (100 ml) were added, and the phases were separated. The aq. phase was extracted with AcOEt (100 ml). The organic phases were pooled and washed with 1 N HCl (2×100 ml) and 1 N NaOH (100 ml). After drying over MgSO₄, the sample was concentrated to yield a yellow oil (10.7 g). The oil was purified by vacuum distillation, and a colorless oil was collected (0.22 torr, 57-59° C., 9.1 g, 62% yield)

### 2-fluoro-N-methoxy-N-methylbenzamide

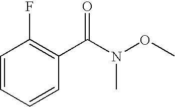

¹H-NMR (300 MHz, CDCl₃) δ 3.34 (s, 3H), 3.54 (br, 3H), 7.10 (m, 1H), 7.19 (m, 1H), 7.42 (m, 2H)

The 6,6-dimethyl-3-methylsulfanyl-6,7-dihydro-5H-thieno[3,4-c]pyridin-4-one is dissolved in THF, and cooled to −78° C. under nitrogen. 2.2 equivalents of a solution of LDA are then added. After stirring at −78° C. for ca. 30 min, (a solution of magnesium chloride or zinc chloride may be advantageous to add at this point) a solution of the desired N-methoxy-N-methyl arylamide in THF is added. The mixture is stirred for 30 min at −78° C., then allowed to warm to rt. After a standard work up and purification the desired product can be isolated.

Step 5.

The product from step 4 can be oxidized with mCPBA as described in the preparation of 3-ethoxycarbonylmethanesulfonyl-6,6-dimethyl-4-oxo-4,5,6,7-tetrahydrobenzo[c]thiophene-1-carboxylic acid ethyl ester or in General Method C, thus yielding the desired compound exemplified below.

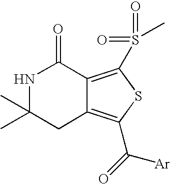

### Example of General Method J

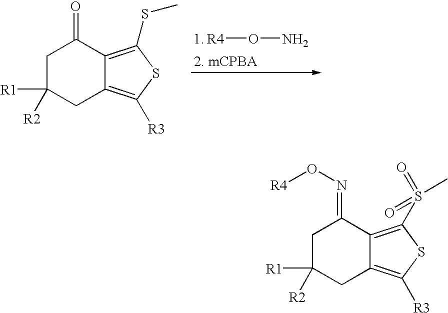

The 4-keto function of the examples described in the invention (1-99) can be converted to oximes by reacting the 4-one intermediates (prior to the oxidation) with an O-substituted hydroxyl amine. The reaction can be carried out in suitable solvents like ethanol or THF, and may or may not be enhanced by the addition of HCl or pyridine. The newly formed oxime containing compound can then be oxidized with mCPBA to either a sulfoxide or a sulfone. Compounds which contain other keto groups would require protection of these keto groups or an alternative synthetic route.

### Example of General Method K

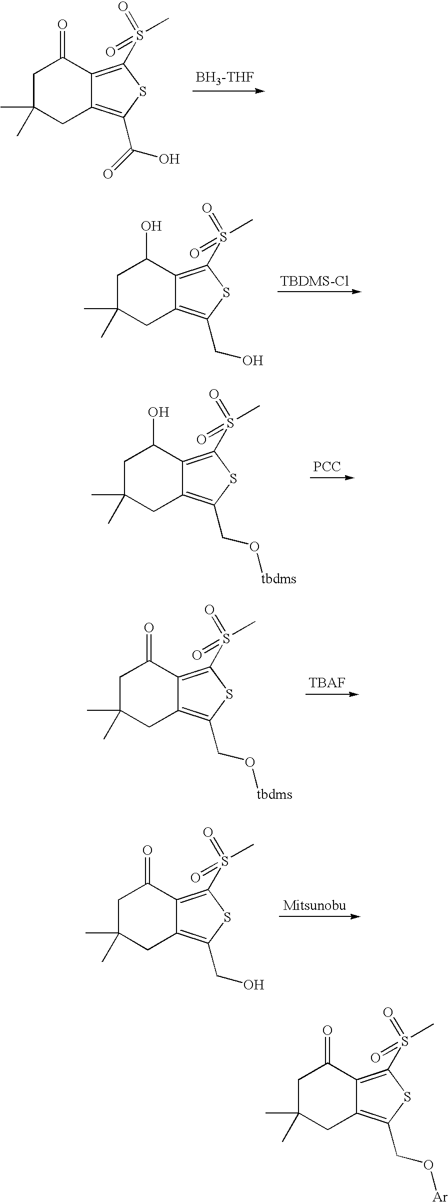

Step 1:

3-Methanesulfonyl-6,6-dimethyl-4-oxo-4,5,6,7-tetrahydrobenzo[c]thiophene-1-carboxylic acid (500 mg, 1.65 mmol) was dissolved in THF (5 ml), placed under nitrogen and cooled to 0° C. A 1 M solution of borane-tetrahydofuran (0.59 ml, 0.36 mmol) in THF was added slowly. The reaction was stirred at 0° C. for 1 h, then removed from the ice bath. After stirring for 3 h at rt, more 1 M borane-tetrahydrofuran (5 ml) in THF was added. The reaction was stirred 16 h, and 10 ml of 1:1 THF/water was added to quench the excess borane. Sat. NaHCO₃ (10 ml) was added, and the phases were separated. The aq. phase was extracted with THF (10 ml). The organic phases were pooled and washed with Sat. NaCl (10 ml), dried over MgSO4, and concentrated to yield a white residue (266 mg).

### 1-Hydroxymethyl-3-methanesulfonyl-6,6-dimethyl-4,5,6,7-tetrahydrobenzo[c]thiophen-4-ol

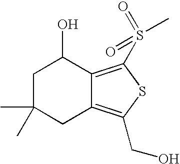

LC-MS (Method A): m/z=313 (M+23); Rₜ=2.25 min.

Step 2:

1-Hydroxymethyl-3-methanesulfonyl-6,6-dimethyl-4,5,6,7-tetrahydrobenzo[c]thiophen-4-ol (1.5 g, 5.2 mmol) was dissolved in DCM (50 ml) under nitrogen. DIEA (3.3 g, 25.8 mmol), DMAP (0.126 g, 1.0 mmol) and tert-butyldimethylsilyl chloride (0.78, 5.2 mmol) were added, and the solution was stirred 16 h at rt. Water (100 ml) was added and the phases were separated. The aqueous phase was extracted with DCM (2×25 ml). The organic phases were pooled, dried over MgSO₄ and concentrated to yield an oil. Purification via flash chromatography (silica, 1:1 heptane/AcOEt) yielded a white crystalline residue (1.24 g, 59% yield)

### 1-(tert-Butyidimethylsilanyloxymethyl)-3-methanesulfonyl-6,6-dimethyl-4,5,6,7-tetrahydrobenzo[c]thiophen-4-ol

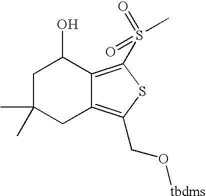

¹H-NMR (300 MHz, CDCl₃) δ 0.13 (s, 6H), 0.90 (s, 3H), 0.92 (s, 9H), 1.12 (s, 3H), 1.65 (m, 1H), 1.92 (dd, 1H), 2.31 (dd, 2H), 3.27 (s, 3H), 4.04 (d, 1H), 4.77 (dd, 2H), 5.11 (m, 1H).

LC-MS (Method A): m/z=427 (M+23); Rₜ=5.18 min.

Step 3:

1-(tert-Butyidimethylsilanyloxymethyl)-3-methanesulfonyl-6,6-dimethyl-4,5,6,7-tetrahydrobenzo[c]thiophen-4-ol (1.24 g, 3.06 mmol) was dissolved in DCM under nitrogen. PCC (1.98 g, 9.19 mmol) and sodium acetate (0.75 g, 9.19 mmol) were added. The solution was stirred for 16 h at rt. Ether (100 ml) was added and the solution was washed with water (2×50 ml), dried over MgSO₄, and concentrated to yield a brown crystalline residue (930 mg, 75% yield).

### 1-(tert-Butyidimethylsilanyloxymethyl)-3-methanesulfonyl-6,6-dimethyl-6,7-dihydro-5H-benzo[c]thiophen-4-one

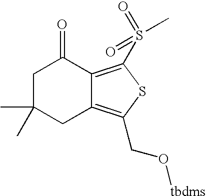

¹H-NMR (300 MHz, CDCl₃) δ 0.13 (s, 6H), 0.93 (s, 9H), 1.08 (s, 6H), 2.49 (s, 2H), 2.58 (s, 2H), 3.48 (s, 3H), 4.80 (s, 2H).

LC-MS (Method A): m/z=403 (M+1); Rₜ=5.05 min.

Step 4:

1-(tert-Butyldimethylsilanyloxymethyl)-3-methanesulfonyl-6,6-dimethyl-6,7-dihydro-5H-benzo[c]thiophen-4-one (930 mg, 2.3 mmol) was dissolved in THF (5 ml), placed under nitrogen, and a 1M solution of TBAF (2.77 ml, 2.77 mmol) was added. After mixing for 1 h at rt, ether (50 ml) was added. The solution was washed with water (2×25 ml), dried over MgSO₄, and concentrated under vacuum to yield a brown crystalline solid (447 mg, 67% yield).

### 1-Hydroxymethyl-3-methanesulfonyl-6,6-dimethyl-6,7-dihydro-5H-benzo [c]thiophen-4-one

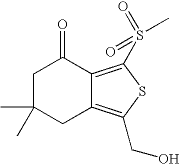

¹H-NMR (300 MHz, CDCl₃): δ 1.08 (s, 6H), 2.50 (s, 2H), 2.68 (s, 2H), 3.48 (s, 3H) 4.81 (br, 2H).

LC-MS (Method A): m/z=289 (M+1); Rₜ=2.20 min.

Step 5:

The final 3-methanesulfonyl-6,6-dimethyl-1-aryloxymethyl-6,7-dihydro-5H-benzo[c]thiophen-4-one can be prepared using the Mitsunobu reaction, which is well described in the literature. Typical reagents would be diisopropylazodicarboxylate and triphenyl phosphine, but there are several other variants available, which are know to the skilled person.

### Example of General Method L

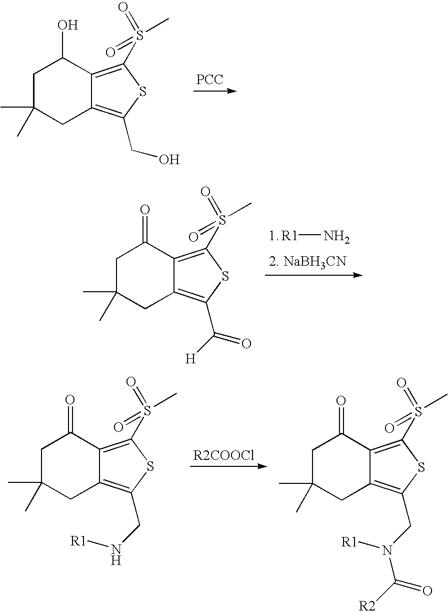

Step 1:

1-Hydroxymethyl-3-methanesulfonyl-6,6-dimethyl-4,5,6,7-tetrahydrobenzo[c]thiophen-4-ol (55 mg, 0.19 mmol) was dissolved in DCM under nitrogen. PCC (245 mg, 1.14 mmol) and sodium acetate (93 mg, 1.14 mmol) were added. The solution was stirred for 16 h at rt. Ether (10 ml) was added and the solution was washed with water (2×5 ml), dried over MgSO₄, and concentrated to yield a residue (34 mg).

### 3-Methanesulfonyl-6,6-dimethyl-4-oxo-4,5,6,7-tetrahydrobenzo[c]thiophene-1-carbaldehyde

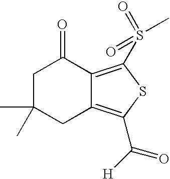

¹H-NMR (300 MHz, CDCl₃) δ 1.14 (s, 6H), 2.58 (s, 2H), 3.15 (s, 2H), 3.53 (s, 3H), 10.07 (s, 1H).

LC-MS (Method A): m/z=287 (M+1); Rₜ=2.74 min.

Step 2:

The aldehyde from step 1 could be used in one of the many reactions know for aldehydes. For example: reductive amination (reaction with an amine followed by reduction to a secondary or tertiary amine).

Step 3:

If primary amines are used in the reductive amination, the resulting secondary amine can be acylated by reacting it with an acid chloride (or a carboxylic acid, HOBt and EDAC), thus forming an amide.

### Example of General Method M

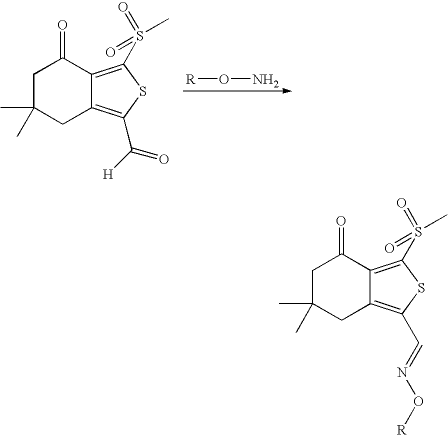

3-Methanesulfonyl-6,6-dimethyl-4-oxo-4,5,6,7-tetrahydrobenzo[c]thiophene-1-carbaldehyde can be reacted with an O-aryl hydroxyl amine or an O-alkyl hydroxyl amine to form 3-methanesulfonyl-6,6-dimethyl-4-oxo-4,5,6,7-tetrahydrobenzo[c]thiophene-1-carbaldehyde O-aryl-oximes or 3-methanesulfonyl-6,6-dimethyl-4-oxo-4,5,6,7-tetrahydrobenzo[c]thiophene-1-carbaldehyde O-alkyl-oximes respectively.

### Example of General Method N

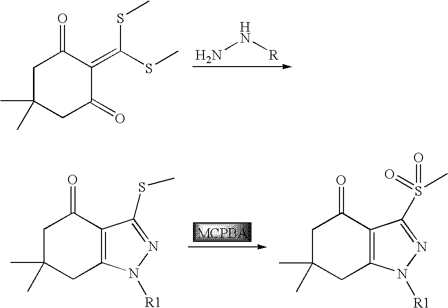

2-(Bismethylsulfanyl-methylene)-5,5-dimethylcyclohexane-1,3-dione can be treated with a hydrazine derivative to give 1-alkyl-6,6-dimethyl-3-methylsulfanyl-1,5,6,7-tetrahydroiondazole-4-one, which subsequently can be oxidized with a suitable oxidizing agent. e.g. mCPBA, to give 1-alkyl-3-methylsufonyl-6,6-dimethyl-1,5,6,7-tetrahydro-indazol-4-one.

### Example of General Method O

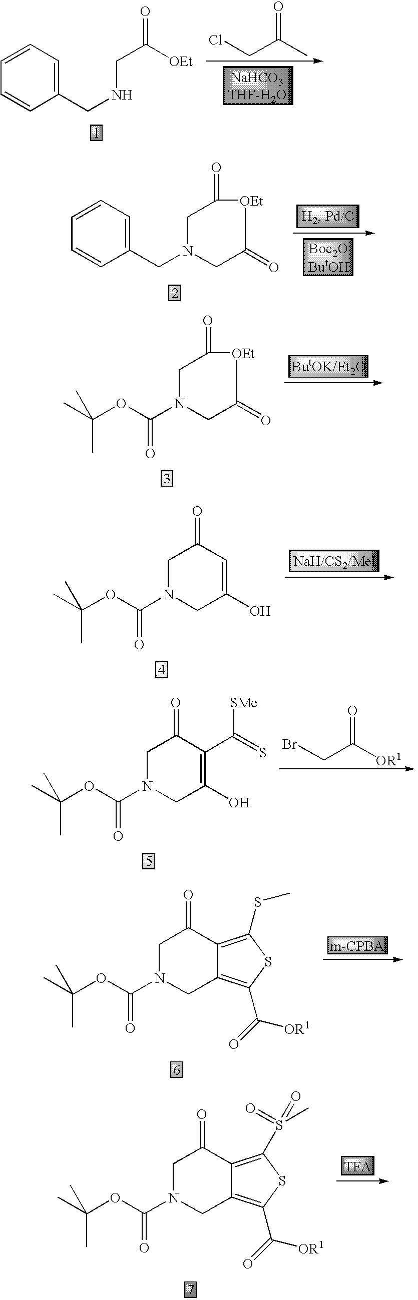

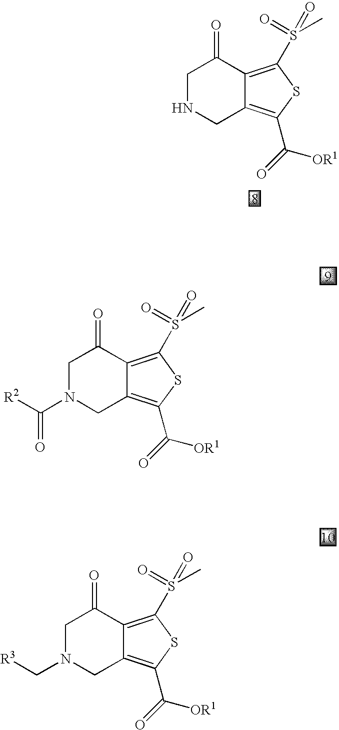

Compounds based on 1-[(1,1-dimethylethoxy)carbonyl]-3,5-dioxopiperidine.

To a heated mixture (50° C.) of ethyl N-benzyl glycinate (100 g, 0.52 mol) and sodium hydrogen carbonate (47.9 g, 0.56 mol) in tetrahydrofuran-water (1 L tetrahydrofuran with 70 mL water) was added chloroacetone (42.1 mL, 0.53 mol) in tetrahydrofuran (100 mL). After completion of the addition, the mixture was stirred for 7 days. Water (500 mL) and heptanes (100 mL) were added. The organic layer was separated off, dried over sodium sulfate (1 h) and concentrated. Ethyl[N-benzyl,N-(2-oxopropyl)]glycinate (128 g, 99%) was obtained and used without further purification.

A stirred mixture of ethyl[N-benzyl,N-(2-oxopropyl)]glycinate (81 g, 0.32 mol), 2-methyl-2-propanol (400 mL), 10% palladium on carbon (5 g) and bis (1,1-dimethylethyl)dicarbonate (70.9 g, 0.32 mol) was treated with a balloon-pressure of hydrogen at room temperature. After 2 days the mixture was filtered through Celite® and concentrated. Column chromatography (SiO₂, ethyl acetate:heptanes=1:3) yielded ethyl[N-((1,1-dimethylethoxy)carbonyl),N-(2-oxopropyl)]glycinate (65 g, 77%).

To a cooled (5° C.) and stirred mixture of potassium 2-methyl-2-propoxide (29.7 g, 0.27 mol) was added a solution of ethyl [N-((1,1-dimethylethoxy)carbonyl),N-(2-oxopropyl)]glycinate (65 g, 0.25 mol) in ethyl ether (200 mL) over a period of 1.5 h. The resulting mixture was stirred for an additional 3 h, and the formed precipitate was filtered off and washed with ethyl ether (2×100 mL). The solid was dissolved in water (150 mL), and acetic acid was added to adjust the pH to 4. The product was filtered, washed with water (2×50 mL) and air dried to furnish 1-[(1,1-dimethylethoxy)carbonyl]-3,5-dioxopiperidine (28 g, 52%).

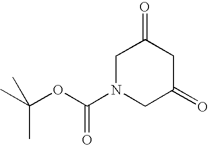

HPLC-MS: m/z: 158 (M+H−56).

¹H-NMR (DMSO-d6): δ 1.48 (9H, s), 4.03 (4H, s), 5.38 (1H, s).

Elaboration of 1-[(1,1-dimethylethoxy)carbonyl]-3,5-dioxopiperidine into 7 may be performed in similar fashion to that described in General Method C by replacing the alphabromo ketone with an alpha-bromo ester. Deprotection using trifluoroacetic acid followed by acylation or reductive alkylation by standard procedures will then provide N-substituted derivatives of 1-methanesulfonyl-7-oxo-4,5,6,7-tetrahydrothieno[3,4-c]pyridine-3-carboxylic acid such as 9 and 10, respectively.

The 1-[(1,1-dimethylethoxy)carbonyl]-3,5-dioxopiperidine could also be used to prepare the types of compounds similar to those described in General Methods B, C, D, E, G, H, K, M, and N.

### Pharmacological Methods

Determination of EC₅₀

Stimulation of cAMP Formation in a Cell Line Expressina the Cloned Human GLP-1 Receptor

In order to demonstrate the efficacy of the GLP-1 agonists, their ability to stimulate formation of cAMP in a cell line expressing the cloned human GLP-1 receptor was tested. The EC₅₀ value was calculated from the dose-response curve. Baby hamster kidney (BHK) cells expressing the human pancreatic GLP-1 receptor were used (Knudsen and Pridal, 1996, Eur. J. Pharm. 318, 429-435).

Two different protocols were used:

Method 1:

Plasma membranes were prepared (Adelhorst et al, 1994, J. Biol. Chem. 269, 6275) by homogenisation in buffer (10 mmol/l Tris-HCl and 30 mmol/l NaCl pH 7.4, containing, in addition, 1 mmol/l dithiothreitol, 5 mg/l leupeptin (Sigma, St. Louis, Mo., USA), 5 mg/l pepstatin (Sigma, St. Louis, Mo., USA), 100 mg/l bacitracin (Sigma, St. Louis, Mo., USA), and 16 mg/l aprotinin (Novo Nordisk A/S, Bagsvaerd, Denmark)). The homogenate was centrifuged on top of a layer of 41 w/v % sucrose. The white band between the two layers was diluted in buffer and centrifuged. Plasma membranes were stored at −80° C. until use.

The assay was carried out in 96-well microtiter plates in a total volume of 200 μl. The resulting concentration in the assay was 50 mmol/l Tris-HCl, pH 7.4, 1 mmol/l EGTA, 1.5 mmol/l MgCl₂, 1.85 mmol/l ATP, 20 μM GTP (guanosine triphosphate), 1 mmol/l 3-isobutyl-1-methylxanthine, 0.01% Tween-20 and 0.1% bovine serum albumin (Reinst, Behringwerke A G, Marburg, Germany). Compounds to be tested for agonist activity were dissolved and diluted in DMSO. GLP-1 was dissolved and diluted in buffer. For GLP-1 test, diluted GLP-1 was added in 35 μl buffer and 10 μl DMSO added extra. For compounds, 10 μl compound in DMSO was added. 1-4 μg plasma membrane in 50 μl buffer was added and the mixture was incubated for 2 hours at 37° C. The reaction was stopped by the addition of 25 μl of 0.5 mol/l HCl. Samples were diluted 5 to 10 fold before analysis for cAMP by a scintillation proximity assay (RPA 538, Amersham, UK). In this assay, GLP-1 was measured with a potency (EC₅₀) of 37±23 μM (n=10).

Method 2:

Membranes were prepared as follows. Suspended cells from one 10 layer cell factory were transferred to 250 ml Sorwall tubes (for GSA rotor and centrifuged at 10.000 g for 10 min at 4° C. 100 ml 25 mM Hepes (pH 7.4), 2.5 mM CaCl₂, 1 mM MgCl₂, 250 mg/l bacitracin, 0.1 mM Pefabloc (homogenizing buffer) were added to the cell pellet which was then homogenised for 2×10 sec. on Ultra-turex (on ice). 100 ml extra homogenising buffer was added and cell nuclei was spun down at 2000 g for 15 min, 4° C. (without brakes). The supernatant containing membranes was transferred to 200 ml tubes (for Sorwall A-621 rotor) and centrifuged at 40.000 g for 45 min at 4° C. The pellet and 100 ml homogenising buffer were homogenised for 2×10 sec. on Ultra-turex (on ice). 100 ml extra homogenising buffer was added and centrifugation continued at 40.000 g for 45 min at 4° C. The membrane pellet was resuspended in 10 ml 25 mM Hepes (pH7.4), 2.5 mM CaCl₂, 1 mM MgCl₂ using Ultra-turex 2×10 sec. (on ice). After protein determination 10 v/v % 25 mM Hepes (pH 7.4), 2.5 mM CaCl₂, 1 mM MgCl₂, 1% BSA, 0.5 mg/ml bacitracin, 2.5 M sucrose was added. The membranes were stored at −80° C. until use.

The assay was carried out in 96-well microtiter plates in a total volume of 200 μl. To 195 μl (50 mmol/l Tris-HCl, pH 7.4, 1 mmol/l EGTA, 1.5 mmol/l MgCl₂, 1.85 mmol/l ATP, 20 μM GTP, 1 mmol/l 3-isobutyl-1-methylxanthine and 0.1% bovine serum albumin (Reinst, Behringwerke A G, Marburg, Germany)), 32 μg plasma membrane protein was added. Compounds to be tested for agonist activity were dissolved and diluted in DMSO. GLP-1 was dissolved and diluted in 0.2% Tween-20. For GLP-1 test, diluted GLP-1 was added in 5 μl 0.2% Tween-20 and 5 μl DMSO added extra. For compounds, 5 μl in DMSO was added and 5 μl 0.2% Tween-20 added extra. The mixture was incubated for 2 hours at 37° C. The reaction was stopped by the addition of 50 μl of 0.5 mol/l HCl. Samples were diluted 5 to 10 fold before analysis for cAMP by a scintillation proximity assay (RPA 538, Amersham, UK). In this assay, GLP-1 was measured with a potency (EC₅₀) of 400±200 pM (n=10).

1. A compound represented by formula I: 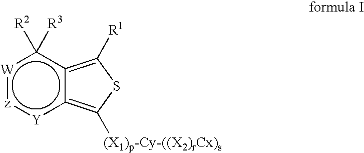
wherein the circle represents optional double bonds anywhere in the ring system;
and R¹ represents —C(O)—R⁴, S(O)₂NHR⁴, C(O)N(R⁴)₂, —SR⁴ or —S(O)R⁴, or —S(O)₂R⁴, wherein R⁴ is hydrogen, C₁₋₆-alkyl, C₂₋₆-alkoxy, C₂₋₆-alkenyl, C₃₋₈-cycloalkyl or C₃₋₈-cycloalkenyl, and when present twice R⁴ can be independently selected from the named substituents;
R² and R³ are independently selected from hydrogen, hydroxy, C₁₋₆-alkyl, hydroxy-C₁₋₆-alkyl C₂₋₆-alkoxy, C₂₋₆-alkylsulfanyl, —NR⁵R⁶, —N═R⁷ or the substituents attached to the same carbon atom together forms a carbonyl or thiocarbonyl group or to ═N—R⁷ or ═N—O—R⁷;
R⁵ and R⁶ are independently selected from hydrogen, C₁₋₆-alkyl, C₂₋₆-alkenyl, C₁₋₆-alkyl-aryl, aryl C₃₋₈-cycloalkyl, C₃₋₈-cycloalkenyl, C₁₋₆-alkanoyl, aroyl, C₃₋₈-cycloalkanoyl;
R⁷ represents hydrogen, C₁₋₆-alkyl, C₂₋₆-alkenyl, aralkyl, aryl, C₃₋₈-cycloalkyl, C₃₋₈-cycloalkenyl, C₁₋₆-alkanoyl, aroyl, C₃₋₈-cycloalkanoyl, C₃₋₈-cycloalkyl, heterocyclyl, heteroaryl and arylene, wherein the rings may optionally be substituted by
hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR⁸, —NR⁸R⁹, —SR⁸, —NR⁸S(O)₂R⁹, —S(O)₂NR⁸R⁹, —S(O)NR⁸R⁹, —S(O)R⁸, —S(O)₂R⁸, —C(O)NR⁸R⁹, —OC(O)NR⁸R⁹, —NR⁸C(O)R⁹, —CH₂C(O)NR⁸R⁹, —OCH₂C(O)NR⁸R⁹, —OC(O)R⁸, —OCH₂C(O)R⁸, —C(O)R⁸ or —C(O)OR⁸, —OCH₂C(O)OR⁸
C₁₋₆-alkyl, C₂₋₆-alkenyl or C₂₋₆-alkynyl,
phenyl
which may optionally be substituted with one or more substituents selected from halogen, —CN, —CF₃, —OCF₃, —NO₂, —OR¹⁰, —NR¹⁰R¹¹ and C₁₋₆-alkyl,
wherein R¹⁰ and R¹¹ independently are hydrogen, C₁₋₆-alkyl, aryl-C₁₋₆-alkyl or aryl,
or R¹⁰ and R¹¹ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds;
W, Y and Z are independently selected from NR¹², or CR¹³R¹⁴
wherein R¹² represents is hydrogen, C₁₋₆-alkyl, C₂₋₆-alkenyl, aralkyl, aryl, C₁₋₆-alkanoyl, aroyl, C₃₋₈-cycloalkanoyl, aryl;
R¹³ and R¹⁴ are independently selected from hydrogen, C₁₋₆-alkyl, C₁₋₆-alkoxy, C₁₋₆-alkylsulfanyl, or R¹³ and R¹⁴ together forms a carbonyl or thiocarbonyl; or the substituents form a double bond in the ring system; The substituents on Z and Y may optionally together form a 5- or 6-membered aromatic ring;
p, r and s are independently 0 or 1
X₁ and X₂ each consist of A-B or B-A
wherein B is the divalent radical of the following selected from C₁₋₆-alkyl, C₂₋₆-alkoxy, C₂₋₆-alkenyl, C₂₋₆-alkynyl, hydroxy-C₁₋₆-alkyl, hydroxy-C₂₋₆-alkenyl, C₁₋₆-alkanoyl, C₂₋₆-alkenoyl;
A is selected from the group consisting of the following: 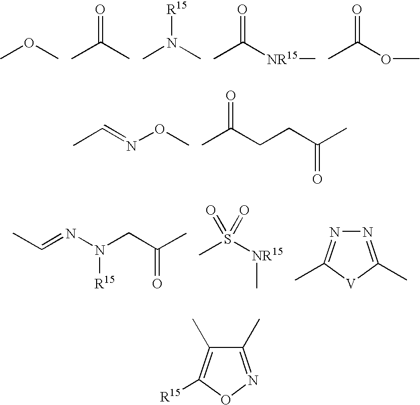
of which all may be attached to B and the ring system above in either direction;
V represents O, S, CHR¹⁵, NR¹⁵
R¹⁵ represents hydrogen, C₁₋₆-alkyl, C₂₋₆-alkenyl, C₁₋₆-alkyl-aryl, aryl, C₃₋₈-cycloalkyl, C₃₋₈-cycloalkenyl, C₁₋₆-alkanoyl, aroyl, C₃₋₈-cycloalkanoyl;
Cy and Cx are independently selected from C₃₋₈-cycloalkyl, heterocyclyl, heteroaryl and arylene, wherein the rings may optionally be substituted by
hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR¹⁷, —NR¹⁷R¹⁸, —SR¹⁷, —NR¹⁷S(O)₂R¹⁸, —S(O)₂NR¹⁷R¹⁸, —S(O)NR¹⁷R¹⁸, —S(O)R¹⁷, —S(O)₂R¹⁷, —C(O)NR¹⁷R¹⁸, —OC(O)NR¹⁷R¹⁸, —NR¹⁷C(O)R¹⁸, —CH₂C(O)NR¹⁷R¹⁸, —OCH₂C(O)NR¹⁷R¹⁸, —OC(O)R¹⁷, —OCH₂C(O)R¹⁷, —C(O)R¹⁷ or —C(O)OR¹⁷, —OCH₂C(O)OR¹⁷
C₁₋₆-alkyl, C₂₋₆-alkenyl or C₂₋₆-alkynyl,
phenyl
which may optionally be further substituted with one or more substituents selected from halogen, —CN, —CF₃, —OCF₃, —NO₂, —OR¹⁷, —NR¹⁷R¹⁸ and C₁₋₆alkyl,
wherein R¹⁷ and R¹⁸ independently are hydrogen, C₁₋₆-alkyl, aryl-C₁₋₆-alkyl or aryl,
or R¹⁷ and R¹⁸ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
wherein Cy may represent the divalent radical of any of the above;
or a pharmaceutically acceptable salt thereof;
with the proviso that when the ring system of formula I together with W, X and Y is selected to represent a 6,6-dimethyl-4,5,6,7-tetrahydrobenzo[c]thiophen-4-one and R¹ represents S—R⁴ then s and p are not simultaneously 0 and in the case of p is 1 and s is o, then X₁-Cy is not pyrrolidin-1yl-carbonyl, N-methyl-cyclohexylaminocarbonyl, homopiperidin-1-ylcarbonyl, morpholin-4-ylcarbonyl, 4-methylpiperazin-1-ylcarbonyl; and if R¹ in the same ring system is SOCH₃ and p and s are both 0 then Cy is not pyrazolyl; and if R¹ in the same ring system is SO₂CH₃ and p is 1 and s is 0 then X₁-Cy does not represent (2-methylcarboxyphenyl)-aminocarbonyl.

2. The compound according to claim 1, wherein formula I is 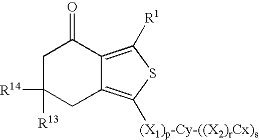
wherein R¹³, R¹⁴, R¹, X₁, X₂, Cy, Cx, p, r and s are as defined in claim 1,
with the proviso that when the ring system of formula I together with W, X and Y is selected to represent a 6,6-dimethyl-4,5,6,7-tetrahydrobenzo[c]thiophen-4-one and R¹ represents S—R⁴ then s and p are not simultaneously 0 and in the case of p is 1 and s is o, then X₁-Cy is not pyrrolidin-1yl-carbonyl, N-methyl-cyclohexylaminocarbonyl, homopiperidin-1-ylcarbonyl, morpholin-4-ylcarbonyl, 4-methylpiperazin-1-ylcarbonyl; and if R¹ in the same ring system is SOCH₃ and p and s are both 0 then Cy is not pyrazolyl; and if R¹ in the same ring system is SO₂CH₃ and p is 1 and s is 0 then X₁-Cy does not represent (2-methylcarboxyphenyl)-aminocarbonyl.

3. The compound according to claim 2, wherein formula I is 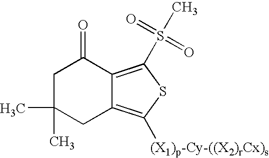
wherein X₁, X₂, Cy, Cx, p, r and s are as defined in claim 1;
with the proviso that if p is 1 and s is 0 then X₁-Cy does not represent (2-methylcarboxyphenyl)-aminocarbonyl.

4. The compound according to claim 1, wherein formula I is 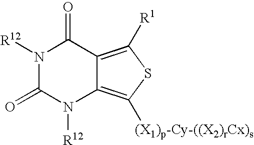
wherein R¹, R¹², X₁, X₂, Cy, Cx, p, r and s are as defined in claim 1.

5. The compound according to claim 1, wherein formula I is 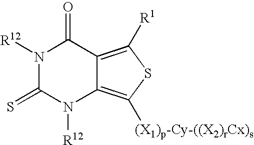
wherein R¹, R¹², X₁, X₂, Cy, Cx, p, r and s are as defined in claim 1.

6. The compound according to claim 1, wherein formula I is 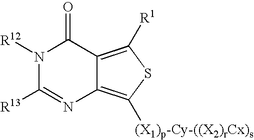
wherein R¹, R¹², R¹³, X₁, X₂, Cy, Cx, p, r and s are as defined in claim 1.

7. The compound according to claim 6, wherein R¹³ is hydrogen, C₁₋₆-alkyl, C₁₋₆-alkoxy, or C₁₋₆-alkylsulfanyl.

8. The compound according to claim 6, wherein R¹³ is C₁₋₆-alkylsulfanyl.

9. The compound according to claim 1, wherein formula I is 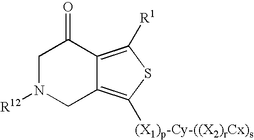
wherein R¹, R¹², X₁, X₂, Cy, Cx, p, r and s are as defined in claim 1.

10. The compound according to claim 9, wherein R¹² is C₁₋₆-alkyl, aralkyl, aryl, C₁₋₆-alkanoyl, aroyl, C₃₋₈-cycloalkanoyl, or aryl.

11. The compound according to claim 1, wherein R¹ represents —S(O)₂R⁴, wherein R⁴ is hydrogen, C₁₋₆-alkyl, C₂₋₆-alkoxy, C₂₋₆-alkenyl, C₃₋₈-cycloalkyl or C₃₋₈-cycloalkenyl.

12. The compound according to claim 11, wherein R⁴ represents C₁₋₆-alkyl.

13. The compound according to claim 11, wherein R⁴ represents methyl.

14. The compound according to claim 1, wherein A of X¹ is selected from 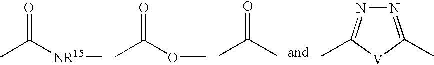
wherein R¹⁵ and V are as defined in claim 1.

15. The compound according to claim 14, wherein A of X¹ is selected from

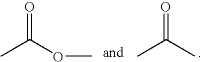

16. The compound according to claim 1, wherein p is 0 or 1.

17. The compound according to claim 1, wherein B of X¹ is C₁₋₆-alkyl.

18. The compound according to claim 17, wherein B of X¹ is selected from the group consisting of methyl, ethyl, n-propyl, isopropyl, butyl, isobutyl, sec-butyl, tert-butyl, n-pentyl, isopentyl, neopentyl, tert-pentyl, n-hexyl, isohexyl and the corresponding divalent derivatives.

19. The compound according to claim 18, wherein C₁₋₆-alkyl is selected from the group consisting of methylene, ethylene, and 1,1-ethylene.

20. The compound according to claim 1, wherein B of X¹ is C₂₋₆-alkylene.

21. The compound according to claim 20, wherein C₂₋₆-alkenyl is selected from the group consisting of vinyl, 1-propenyl, 2-propenyl, iso-propenyl, 1,3-butadienyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 2,4-hexadienyl, and 5-hexenyl.

22. The compound according to claim 21, wherein C₂₋₆-alkenyl is selected from 1-propenyl, 2-propenyl or iso-propenyl.

23. The compound according to claim 1, wherein Cx or Cy is heteroaryl or divalent radicals thereof.

24. The compound according to claim 23, wherein Cx or Cy is selected from the group consisting of furyl, thienyl, pyrrolyl, 2,5-oxadiazolyl, 1,2,5-thiadiazolyl, tetrazolyl, thiazolyl, oxazolyl, isoxazolyl, isothiazolyl, pyridyl, 2,3-dihydrobenzofuiranyl, benzodioxanyl, benzoxanyl, methylenedioxybenzene, diphenyleneoxide, pyrrolinyl, pyrazolinyl, indolinyl, oxazolidinyl, oxazolinyl or oxazepinyl or divalent radicals thereof.

25. The compound according to claim 24, wherein Cx or Cy is selected from thienyl, thiazolyl, tetrazolyl, pyridyl, oxazolyl, 2,3-dihydrobenzofuranyl, benzodioxanyl, benzoxanyl, methylenedioxybenzene, diphenyleneoxide, pyrrolinyl, pyrazolinyl, indolinyl, oxazolidinyl, oxazolinyl or oxazepinyl or divalent radicals thereof.

26. The compound according to claim 1, wherein Cx or Cy is arylene or aryl.

27. The compound according to claim 26, wherein Cx or Cy is selected from the group consisting of phenylene, biphenylylene, naphthylene, anthracenylene, phenanthrenylene, fluorenylene, indenylene, pentalenylene, azulenylene,1,2,3,4-tetrahydronaphthylene, and 1,4-dihydronaphthylene.

28. The compound according to claim 27, wherein Cx or Cy represents phenylene.

29. The compound according to claim 28, wherein Cx or Cy represents.

30. The compound according to claim 29, wherein Cx or Cy is selected from the group consisting of phenyl, biphenylyl, naphthyl, anthracenyl, phenanthrenyl, fluorenyl, indenyl, pentalenyl, indanyl, 1,2,3,4-tetrahydronaphthyl, and 1,4-dihydronaphthyl.

31. The compound according to claim 30, wherein Cx or Cy is selected form the group consisting of phenyl, naphthyl 1,2,3,4-tetrahydronaphthyl and indanyl.

32. The compound according to claim 1 wherein Cx or Cy is C₃₋₈-cycloalkyl.

33. The compound according to claim 32, wherein Cx or Cy is cyclohexyl.

34. The compound according to claim 1, wherein Cx is phenyl, benzodioxanyl, 2-benzodioxanyl, chromanyl, indanyl, 1-indanyl, 2-indanyl, cyclohexyl, benzodioxolyl, naphthyl, oxazolyl, dibenzofuranyl, isoxazolyl, pyridyl, 1,1-dioxo-1H-benzo[b]thiophenyl, aminothiazolyl, tetrazolyl or 1,3,4-oxadiazolyl.

35. The compound according to claim 1, wherein Cy is selected from the group consisting of phenyl, cyclohexyl, naphthyl, benzofuranyl, and benzyl.

36. The compound according to claim 1, wherein Cx or Cy is substituted one or more times by substituents selected independently from the group consisting of hydrogen, halogen, —CN, —CF₃, —OCF₃, —OCHF₂, —NO₂, —OR⁸—C(O)OR⁸, —OCH₂C(O)OR⁸, C₁₋₆-alkyl, and phenyl.

37. The compound according to claim 1, wherein X₂ is selected from the group consisting of C₁₋₆-alkyl 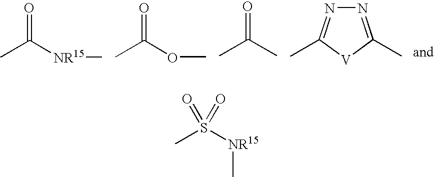
wherein R¹⁵ and V are as defined in claim 1.

38. (canceled)

39. (canceled)

40. (canceled)

41. (canceled)

42. A pharmaceutical composition comprising a compound according to claim 1 together with pharmaceutically acceptable carriers and diluents.

43. A method of treating or preventing a disease or disorder wherein GLP-1 agonistic action is beneficial, comprising administering to a subject in need of such treatment a pharmaceutically effective amount of a compound according to claim 1.

44. (canceled)

45. (canceled)
